(19) United States
(12) Patent Application Publication
(10) Pub. No.: US 2021/0085785 A1
(43) Pub. Date: Mar. 25, 2021

(54) TREATING CANCER BY BLOCKING THE INTERACTION OF VISTA AND ITS BINDING PARTNER

(71) Applicant: TrueBinding, Inc., Menlo Park, CA (US)

(72) Inventors: Dongxu SUN, Los Altos, CA (US); Yan WANG, Concord, CA (US); Catherine A. GORDON, Fremont, CA (US); Yi CHAI, Cupertino, CA (US); Samuel A.F. WILLIAMS, Burlingame, CA (US)

(21) Appl. No.: 16/971,559

(22) PCT Filed: Feb. 22, 2019
(86) PCT No.: PCT/US19/19186

§ 371 Date: Aug. 20, 2020

Related U.S. Application Data
(60) Provisional application No. 62/634,649, filed on Feb. 23, 2018.

(51) Int. Cl. A61K 39/395; C07K 16/28; A61P 35/00; A61K 45/06

(52) U.S. Cl. CPC A61K 39/3955; C07K 16/2803; A61P 35/00; A61K 45/06; C07K 2317/76; C07K 2317/31; C07K 2317/55; C07K 2317/22; C07K 2317/565; C07K 2317/56; C07K 2317/569

(57) ABSTRACT

Disclosed herein are antibodies that specifically bind to LRIG1 and methods of use thereof. In some embodiments, also described herein are methods of inducing immune activation or promoting B cell or Natural Killer cell proliferation with an antibody that specifically binds to LRIG1.

### CROSS-REFERENCE

This application is a U.S. National Phase of PCT International App. No. PCT/US2019/019186, filed on Feb. 22, 2019, designating the United States of America and published in the English language, which claims the benefit of U.S. Provisional Patent Application No. 62/634,649, filed on Feb. 23, 2018, each of which is incorporated herein by reference in its entirety.

### REFERENCE TO SEQUENCE LISTING

The present application is being filed along with a Sequence Listing in electronic format. The Sequence Listing is provided as a file entitled IMMUT009NP.TXT, which was created and last modified on Aug. 20, 2020, which is 51.393 bytes in size. The information in the electronic Sequence Listing is hereby incorporated by reference in its entirety.

### SUMMARY OF THE DISCLOSURE

Disclosed herein, in some embodiments, are methods of using anti-LRIG1 antibodies to induce immune activation. Further disclosed herein, in some embodiments, are methods of using anti-LRIG1 antibodies to promote B cell, T cell, and/or Natural Killer (NK) cell proliferation.

Disclosed herein, in certain embodiments, is a method of disrupting an interaction between VISTA and LRIG1, comprising: contacting a plurality of cells comprising a LRIG1-expressing cell, a VISTA-expressing cell, or a combination thereof with an antibody that specifically binds to LRIG1. In some embodiments, the LRIG1-VISTA interaction is reduced to less than 80%, less than 78%, less than 70%, less than 72%, less than 66%, less than 60%, less than 56%, less than 54%, less than 52%, less than 50%, less than 44%, less than 43%, less than 40%, less than 30%, less than 29%, less than 27%, less than 21%, less than 20%, less than 19%, less than 17%, less than 10%, less than 5%, or less than 1%. In some embodiments, the interaction occurs at one or more residues of LRIG1 selected from region 245-260, wherein the residue positions correspond to positions 245-260 of SEQ ID NO: 2. In some embodiments, the interaction occurs at one or more residues of VISTA selected from region 78-90 or 68-92, wherein the residue positions correspond to positions 78-90 or 68-92 of SEQ ID NO: 4. In some embodiments, the antibody binds to at least one amino acid residue within Peptide 54 or Peptide 61. In some embodiments, the antibody comprises a kD of less than 1 nM, 1.2 nM, 2 nM, 5 nM, 10 nM, 13.5 nM, 15 nM, 20 nM, 25 nM, or 30 nM. In some embodiments, the antibody comprises a humanized antibody. In some embodiments, the antibody comprises a full-length antibody or a binding fragment thereof. In some embodiments, the antibody comprises a bispecific antibody or a binding fragment thereof. In some embodiments, the antibody comprises a monovalent Fab′, a divalent Fab2, a single-chain variable fragment (scFv), a diabody, a minibody, a nanobody, a single-domain antibody (sdAb), or a camelid antibody or binding fragment thereof. In some embodiments, the antibody is a humanized antibody comprising six complementarity-determining regions (CDRs) SEQ ID NOs: 81-86. In some embodiments, the humanized antibody comprises a heavy chain variable region (VH) selected from SEQ ID NOs: 87 and 88. In some embodiments, the humanized antibody comprises a light chain variable region (VL) selected from SEQ ID NOs: 89 and 90. In some embodiments, the antibody is mab2, mab4, mab5, or mab6. In some embodiments, the antibody comprises an IgG framework. In some embodiments, the antibody comprises an IgG1, IgG2, or IgG4 framework.

Disclosed herein, in certain embodiments, is a method of inducing immune activation, comprising: contacting a plurality of cells comprising a LRIG1-expressing cell with an antibody under conditions to effect production of a cytokine, thereby inducing immune activation, wherein the antibody specifically binds to LRIG1. In some embodiments, the plurality of cells further comprises a VISTA expressing cell. In some embodiments, the anti-LRIG1 antibody further inhibits or disrupts an interaction of LRIG1 and VISTA. In some embodiments, the LRIG1-VISTA interaction is reduced to less than 80%, less than 78%, less than 70%, less than 72%, less than 66%, less than 60%, less than 56%, less than 54%, less than 52%, less than 50%, less than 44%, less than 43%, less than 40%, less than 30%, less than 29%, less than 27%, less than 21%, less than 20%, less than 19%, less than 17%, less than 10%, less than 5%, or less than 1%. In some embodiments, the interaction occurs at one or more residues of LRIG1 selected from region 245-260, wherein the residue positions correspond to positions 245-260 of SEQ ID NO: 2. In some embodiments, the interaction occurs at one or more residues of VISTA selected from region 78-90 or 68-92, wherein the residue positions correspond to positions 78-90 or 68-92 of SEQ ID NO: 4. In some embodiments, the antibody binds to at least one amino acid residue within Peptide 54 or Peptide 61. In some embodiments, the antibody comprises a kD of less than 1 nM, 1.2 nM, 2 nM, 5 nM, 10 nM, 13.5 nM, 15 nM, 20 nM, 25 nM, or 30 nM. In some embodiments, the antibody comprises a humanized antibody. In some embodiments, the antibody comprises a full-length antibody or a binding fragment thereof. In some embodiments, the antibody comprises a bispecific antibody or a binding fragment thereof. In some embodiments, the antibody comprises a monovalent Fab′, a divalent Fab2, a single-chain variable fragment (scFv), a diabody, a minibody, a nanobody, a single-domain antibody (sdAb), or a camelid antibody or binding fragment thereof. In some embodiments, the antibody is a humanized antibody comprising six complementarity-determining regions (CDRs) SEQ ID NOs: 81-86. In some embodiments, the humanized antibody comprises a heavy chain variable region (VH) selected from SEQ ID NOs: 87 and 88. In some embodiments, the humanized antibody comprises a light chain variable region (VL) selected from SEQ ID NOs: 89 and 90. In some embodiments, the antibody is mab2, mab4, mab5, or mab6. In some embodiments, the antibody comprises an IgG framework. In some embodiments, the antibody comprises an IgG1, IgG2, or IgG4 framework. In some embodiments, the cytokine is an interferon. In some embodiments, the interferon is IFNγ. In some embodiments, the antibody results in IFNγ production higher than an isotype antibody. In some embodiments, the immune activation comprises a proliferation of CD3+T lymphocytes, CD4+T helper cells, CD8+ cytotoxic T cells, B cells, Natural Killer cells, or a combination thereof. In some embodiments, the immune activation comprises an increase in M1 macrophage population within the plurality of cells. In some embodiments, the immune activation comprises a decrease in M2 macrophage population within the plurality of cells.

Disclosed herein, in certain embodiments, is a method of reducing tumor cells within a tumor microenvironment (TME) in a subject, comprising contacting a plurality of cells located within the TME with an antibody that specifically binds to LRIG1. In some embodiments, the tumor cells are reduced by at least 5%, 10%, 15%, 20%, 25%, 30%, 35%, 40%, 45%, 50%, 60%, 70%, 80%, or 90%. In some embodiments, the subject is diagnosed with a cancer. In some embodiments, the cancer is a solid tumor. In some embodiments, the cancer is breast cancer, colorectal cancer, kidney cancer, liver cancer, or lung cancer. In some embodiments, the cancer is a hematologic malignancy. In some embodiments, the cancer is a metastatic cancer. In some embodiments, the cancer is a relapsed or refractory cancer. In some embodiments, the antibody is formulated for systemic administration. In some embodiments, the antibody is formulated for parenteral administration. In some embodiments, the antibody is administered in combination with an additional therapeutic agent. In some embodiments, the antibody and the additional therapeutic agent are administered simultaneously. In some embodiments, the antibody and the additional therapeutic agent are administered sequentially. In some embodiments, the antibody is administered prior to administering the additional therapeutic agent. In some embodiments, the antibody is administered after administering the additional therapeutic agent. In some embodiments, the additional therapeutic agent comprises an immune checkpoint modulator. In some embodiments, the additional therapeutic agent comprises a chemotherapeutic agent, targeted therapeutic agent, hormonal therapeutic agent, or a stem cell-based therapeutic agent. In some embodiments, the antibody is administered either prior to or after surgery. In some embodiments, the antibody is administered in conjunction with, before, or after radiation therapy. In some embodiments, the anti-LRIG1 antibody further inhibits or disrupts an interaction of LRIG1 and VISTA. In some embodiments, the LRIG1-VISTA interaction is reduced to less than 80%, less than 78%, less than 70%, less than 72%, less than 66%, less than 60%, less than 56%, less than 54%, less than 52%, less than 50%, less than 44%, less than 43%, less than 40%, less than 30%, less than 29%, less than 27%, less than 21%, less than 20%, less than 19%, less than 17%, less than 10%, less than 5%, or less than 1%. In some embodiments, the interaction occurs at one or more residues of LRIG1 selected from region 245-260, wherein the residue positions correspond to positions 245-260 of SEQ ID NO: 2. In some embodiments, the interaction occurs at one or more residues of VISTA selected from region 78-90 or 68-92, wherein the residue positions correspond to positions 78-90 or 68-92 of SEQ ID NO: 4. In some embodiments, the antibody binds to at least one amino acid residue within Peptide 54 or Peptide 61. In some embodiments, the antibody comprises a kD of less than 1 nM, 1.2 nM, 2 nM, 5 nM, 10 nM, 13.5 nM, 15 nM, 20 nM, 25 nM, or 30 nM. In some embodiments, the antibody comprises a humanized antibody. In some embodiments, the antibody comprises a full-length antibody or a binding fragment thereof. In some embodiments, the antibody comprises a bispecific antibody or a binding fragment thereof. In some embodiments, the antibody comprises a monovalent Fab′, a divalent Fab2, a single-chain variable fragment (scFv), a diabody, a minibody, a nanobody, a single-domain antibody (sdAb), or a camelid antibody or binding fragment thereof. In some embodiments, the antibody is a humanized antibody comprising six complementarity-determining regions (CDRs) SEQ ID NOs: 81-86. In some embodiments, the humanized antibody comprises a heavy chain variable region (VH) selected from SEQ ID NOs: 87 and 88. In some embodiments, the humanized antibody comprises a light chain variable region (VL) selected from SEQ ID NOs: 89 and 90. In some embodiments, the antibody is mab2, mab4, mab5, or mab6. In some embodiments, the antibody comprises an IgG framework. In some embodiments, the antibody comprises an IgG1, IgG2, or IgG4 framework. In some embodiments, the method further comprises inducing immune activation. In some embodiments, the immune activation comprises production of a cytokine. In some embodiments, the cytokine is an interferon, optionally IFNγ. In some embodiments, the immune activation comprises a proliferation of CD3+T lymphocytes, CD4+T helper cells, CD8+ cytotoxic T cells, B cells, Natural Killer cells, or a combination thereof. In some embodiments, the immune activation comprises an increase in M1 macrophage population within the plurality of cells. In some embodiments, the immune activation comprises a decrease in M2 macrophage population within the plurality of cells. In some embodiments, the subject is a human.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings below. The patent application file contains at least one drawing executed in color. Copies of this patent application publication with color drawing(s) will be provided by the Office upon request and payment of the necessary fee.

FIG. 1A-FIG. 1C illustrate the results of co-immunoprecipitation assay indicating that human LRIG1 (hLRIG1) specifically pulled down human VISTA. FIG. 1A and FIG. 1B show expression of LRIG1 and VISTA, respectively, in 293T cells co-transfected with a plasmid encoding a HA-tagged hVISTA and a plasmid encoding Flag-tagged hLRIG1. FIG. 1C shows that LRIG1 pulled down the-VISTA in the co-transfected 293T cells.

FIG. 2 shows the results of ELISA assays performed to assess the binding of hLRIG1 to VISTA in the presence or absence of anti LRIG1 mAb (IMT-300).

FIG. 3A-FIG. 3B shows the results of flow cytometry analysis of LRIG1 expression on activated human peripheral blood mononuclear cells (PBMCs) (FIG. 3B) and inactivated PBMCs (FIG. 3A).

FIG. 4 shows the measurements of IFNgamma production in Mixed Lymphocyte Reaction assays, in which human M2 macrophages from one donor were mixed with human CD4 T cells from another donor and were treated with 10 ug/ml control IgG, hPD1 blocking antibody EH12 (BD bioscience), hLRIG1 mAb IMT300 (also referred to herein as mab4), or the combination of hPD1 and LRIG1 antibodies for 8 days.

FIG. 5 shows an ELISA assessment of LRIG1-VISTA interaction blockade by LRIG1 binding antibodies. Percent of LRIG1-VISTA binding in the absence of antibody is shown.

FIG. 6 shows an ELISA assessment of anti-LRIG1 antibody binding to peptide fragments of LRIG1.

FIG. 7A-FIG. 7C show MALDI-MS identification of LRIG1 and VISTA regions mediating the interaction between LRIG1 and VISTA. FIG. 7A and FIG. 7C illustrate the interaction site and residues within the site. FIG. 7B illustrates a crystal structure of LRIG1 highlighting the region mediating the interaction.

FIG. 8 shows a graph evaluating anti-LRIG1 antibody on anti-tumor activity in SCLC xenograft tumors in mice engrafted with human immune systems.

### DETAILED DESCRIPTION OF THE DISCLOSURE

Tumors are often associated with an immune infiltrate as part of the reactive stroma that is enriched for macrophages. Tumor-associated macrophages (TAMs) play an important role in facilitating tumor growth by promoting neovascularization and matrix degradation. When associated with tumors, macrophages demonstrate functional polarization towards one of two phenotypically different subsets of macrophages: M1 macrophages or M2 macrophages. M1 macrophages are known to produce pro-inflammatory cytokines and play an active role in cell destruction, while M2 macrophages primarily scavenge debris and promote angiogenesis and wound repair. Consequently, many tumors with a high number of TAMs have an increased tumor growth rate, local proliferation, and distant metastasis. The M2 macrophage population is phenotypically similar to the TAM population that promotes tumor growth and development. In addition to expressing VISTA, M2 macrophages, in some cases, also express one or more cell surface markers selected from the group consisting of CD206, IL-4r, IL-1ra, decoy IL-1r11, IL-10r, CD23, macrophage scavenging receptors A and B, Ym-1, Ym-2, Low density receptor-related protein 1 (LRP1), IL-6r, CXCR1/2, CD136, CD14, CD1a, CD1b, CD93, CD226, (FcγR) and PD-L1.

VISTA (V-domain Ig suppressor of T cell activation) is expressed in high levels in myeloid cells, which include monocytes, macrophages, neutrophils, basophils, eosinophils, erythrocytes, dendritic cells, megakaryocytes and platelets. VISTA levels are heightened within the tumor microenvironment.

LRIG1 (Leucine-rich repeats and immunoglobulin-like domains protein 1) is a transmembrane protein that has been shown to interact with receptor tyrosine kinases of the EGFR-family, MET and RET. In some instances, LRIG1 has found to be a tumor suppressor and negative regulator of receptor tyrosine kinases.

In some embodiments, disclosed herein are anti-LRIG1 antibodies that interfere with the interaction between VISTA and LRIG1 and activate an immune response. In some instances, these anti-LRIG1 antibodies are used to treat cancers or other diseases that could benefit from activation of immune response.

### Methods of Use

In certain embodiments, disclosed herein are methods of inducing immune activation, comprising contacting an anti-LRIG1 antibody to a plurality of cells comprising a VISTA-expressing cell, a LRIG1 expressing cell, or a combination thereof.

In some cases, the LRIG1-expressing cell upon binding to the anti-LRIG1 antibody expresses a cytokine which induces immune activation. In some cases, the cytokine is an interferon. In some cases, the interferon is IFNγ. In some cases, the IFNγ production is 110%, 120%, 130%, 140%, 150%, 160%, 170%, 180%, 190%, 200%, 300%, 400%, 500%, 600%, or more of IFNγ production by an isotype antibody. In some cases, the IFNγ production is 150% of IFNγ production by an isotype antibody. In some cases, the IFNγ production is 160% of IFNγ production by an isotype antibody. In some cases, the IFNγ production is 170% of IFNγ production by an isotype antibody. In some cases, the IFNγ production is 180% of IFNγ production by an isotype antibody. In some cases, the IFNγ production is 190% of IFNγ production by an isotype antibody. In some cases, the IFNγ production is 200% of IFNγ production by an isotype antibody. In some cases, the IFNγ production is more than 200% of IFNγ production by an isotype antibody. In some cases, the IFNγ production is more than 300% of IFNγ production by an isotype antibody. In some cases, the IFNγ production is more than 400% of IFNγ production by an isotype antibody. In some cases, the IFNγ production is more than 500% of IFNγ production by an isotype antibody. In some cases, the cytokine is an interleukin. In some cases, the interleukin is IL-2.

In some cases, the immune activation comprises a proliferation of CD3+T lymphocytes, CD4+T helper cells, CD8+ cytotoxic T cells, B cells, Natural Killer (NK) cells, or a combination thereof. In some cases, the immune activation comprises a proliferation of CD3+T lymphocytes. In some cases, the immune activation comprises a proliferation of CD4+T helper cells. In some cases, the immune activation comprises a proliferation of CD8+ cytotoxic T cells. In some cases, the immune activation comprises a proliferation of B cells. In some cases, the immune activation comprises a proliferation of NK cells. In some cases, the immune activation comprises a proliferation of B cells and NK cells.

In some cases, the immune activation comprises an increase in M1 macrophage population within the plurality of cells. In some cases, the immune activation comprises a decrease in M2 macrophage population within the plurality of cells. In some cases, the immune activation comprises an increase in M1 macrophage population within the plurality of cells and a decrease in M2 macrophage population within the plurality of cells.

In some cases, an anti-LRIG1 antibody binds to LRIG1 and disrupts an interaction between VISTA and LRIG1. In some cases, disruption of an interaction between VISTA and LRIG1 includes partial inhibition of interaction between VISTA and LRIG1. In some cases, disruption of an interaction between VISTA and LRIG1 includes complete inhibition of interaction between VISTA and LRIG1. In some cases, the anti-LRIG1 antibody binds to LRIG1 and reduces an interaction between VISTA and LRIG1. In some cases, the VISTA-LRIG1 interaction is reduced to less than 80%, less than 78%, less than 70%, less than 72%, less than 66%, less than 60%, less than 56%, less than 54%, less than 52%, less than 50%, less than 44%, less than 43%, less than 40%, less than 30%, less than 29%, less than 27%, less than 21%, less than 20%, less than 19%, less than 17%, less than 10%, less than 5%, or less than 1%. In some cases, the LRIG1-VISTA interaction is reduced to less than 70%. In some cases, the VISTA-LRIG1 interaction is reduced to less than 60%. In some cases, the VISTA-LRIG1 interaction is reduced to less than 59%. In some cases, the VISTA-LRIG1 interaction is reduced to less than 50%. In some cases, the VISTA-LRIG1 interaction is reduced to less than 44%. In some cases, the VISTA-LRIG1 interaction is reduced to less than 43%. In some cases, the VISTA-LRIG1 interaction is reduced to less than 40%. In some cases, the VISTA-LRIG1 interaction is reduced to less than 34%. In some cases, the VISTA-LRIG1 interaction is reduced to less than 30%. In some cases, the VISTA-LRIG1 interaction is reduced to less than 21%. In some cases, the VISTA-LRIG1 interaction is reduced to less than 20%. In some cases, the VISTA-LRIG1 interaction is reduced to less than 14%. In some cases, the VISTA-LRIG1 interaction is reduced to less than 10%. In some cases, the VISTA-LRIG1 interaction is reduced to less than 7%. In some cases, the VISTA-LRIG1 interaction is reduced to less than 5%. In some cases, the VISTA-LRIG1 interaction is reduced to less than 4%. In some cases, the VISTA-LRIG1 interaction is reduced to less than 1%.

In some cases, the interaction between VISTA and LRIG1 occurs at one or more residues of LRIG1 selected from region 245-260, wherein the residue positions correspond to positions 245-260 of SEQ ID NO: 2. In some cases, the interaction between VISTA and LRIG1 occurs at residue 245, wherein the residue position corresponds to position 245 of SEQ ID NO: 2. In some cases, the interaction between VISTA and LRIG1 occurs at residue 246, wherein the residue position corresponds to position 246 of SEQ ID NO: 2. In some cases, the interaction between VISTA and LRIG1 occurs at residue 247, wherein the residue position corresponds to position 247 of SEQ ID NO: 2. In some cases, the interaction between VISTA and LRIG1 occurs at residue 248, wherein the residue position corresponds to position 248 of SEQ ID NO: 2. In some cases, the interaction between VISTA and LRIG1 occurs at residue 249, wherein the residue position corresponds to position 249 of SEQ ID NO: 2. In some cases, the interaction between VISTA and LRIG1 occurs at residue 250, wherein the residue position corresponds to position 250 of SEQ ID NO: 2. In some cases, the interaction between VISTA and LRIG1 occurs at residue 251, wherein the residue position corresponds to position 251 of SEQ ID NO: 2. In some cases, the interaction between VISTA and LRIG1 occurs at residue 252, wherein the residue position corresponds to position 252 of SEQ ID NO: 2. In some cases, the interaction between VISTA and LRIG1 occurs at residue 253, wherein the residue position corresponds to position 253 of SEQ ID NO: 2. In some cases, the interaction between VISTA and LRIG1 occurs at residue 254, wherein the residue position corresponds to position 254 of SEQ ID NO: 2. In some cases, the interaction between VISTA and LRIG1 occurs at residue 255, wherein the residue position corresponds to position 255 of SEQ ID NO: 2. In some cases, the interaction between VISTA and LRIG1 occurs at residue 256, wherein the residue position corresponds to position 256 of SEQ ID NO: 2. In some cases, the interaction between VISTA and LRIG1 occurs at residue 257, wherein the residue position corresponds to position 257 of SEQ ID NO: 2. In some cases, the interaction between VISTA and LRIG1 occurs at residue 258, wherein the residue position corresponds to position 258 of SEQ ID NO: 2. In some cases, the interaction between VISTA and LRIG1 occurs at residue 259, wherein the residue position corresponds to position 259 of SEQ ID NO: 2. In some cases, the interaction between VISTA and LRIG1 occurs at residue 260, wherein the residue position corresponds to position 260 of SEQ ID NO: 2. In some cases, LRIG1 is human LRIG1.

In some cases, the interaction between LRIG1 and VISTA occurs at one or more residues of VISTA selected from region 78-90 or 68-92, wherein the residue positions correspond to positions 78-90 or 68-92 of SEQ ID NO: 4. In some cases, the interaction between LRIG1 and VISTA occurs at one or more residues of VISTA from region 78-90, wherein the residue positions correspond to positions 78-90 of SEQ ID NO: 4. In some cases, the interaction between LRIG1 and VISTA occurs at one or more residues of VISTA from region 68-92, wherein the residue positions correspond to positions 68-92 of SEQ ID NO: 4. In some cases, the interaction between LRIG1 and VISTA occurs at residue 68, wherein the residue position corresponds to positions 68 of SEQ ID NO: 4. In some cases, the interaction between LRIG1 and VISTA occurs at residue 69, wherein the residue position corresponds to positions 69 of SEQ ID NO: 4. In some cases, the interaction between LRIG1 and VISTA occurs at residue 70, wherein the residue position corresponds to positions 70 of SEQ ID NO: 4. In some cases, the interaction between LRIG1 and VISTA occurs at residue 71, wherein the residue position corresponds to positions 71 of SEQ ID NO: 4. In some cases, the interaction between LRIG1 and VISTA occurs at residue 72, wherein the residue position corresponds to positions 72 of SEQ ID NO: 4. In some cases, the interaction between LRIG1 and VISTA occurs at residue 73, wherein the residue position corresponds to positions 73 of SEQ ID NO: 4. In some cases, the interaction between LRIG1 and VISTA occurs at residue 74, wherein the residue position corresponds to positions 74 of SEQ ID NO: 4. In some cases, the interaction between LRIG1 and VISTA occurs at residue 75, wherein the residue position corresponds to positions 75 of SEQ ID NO: 4. In some cases, the interaction between LRIG1 and VISTA occurs at residue 76, wherein the residue position corresponds to positions 76 of SEQ ID NO: 4. In some cases, the interaction between LRIG1 and VISTA occurs at residue 77, wherein the residue position corresponds to positions 77 of SEQ ID NO: 4. In some cases, the interaction between LRIG1 and VISTA occurs at residue 78, wherein the residue position corresponds to positions 78 of SEQ ID NO: 4. In some cases, the interaction between LRIG1 and VISTA occurs at residue 79, wherein the residue position corresponds to positions 79 of SEQ ID NO: 4. In some cases, the interaction between LRIG1 and VISTA occurs at residue 80, wherein the residue position corresponds to positions 80 of SEQ ID NO: 4. In some cases, the interaction between LRIG1 and VISTA occurs at residue 81, wherein the residue position corresponds to positions 81 of SEQ ID NO: 4. In some cases, the interaction between LRIG1 and VISTA occurs at residue 82, wherein the residue position corresponds to positions 82 of SEQ ID NO: 4. In some cases, the interaction between LRIG1 and VISTA occurs at residue 83, wherein the residue position corresponds to positions 83 of SEQ ID NO: 4. In some cases, the interaction between LRIG1 and VISTA occurs at residue 84, wherein the residue position corresponds to positions 84 of SEQ ID NO: 4. In some cases, the interaction between LRIG1 and VISTA occurs at residue 85, wherein the residue position corresponds to positions 85 of SEQ ID NO: 4. In some cases, the interaction between LRIG1 and VISTA occurs at residue 86, wherein the residue position corresponds to positions 86 of SEQ ID NO: 4. In some cases, the interaction between LRIG1 and VISTA occurs at residue 87, wherein the residue position corresponds to positions 87 of SEQ ID NO: 4. In some cases, the interaction between LRIG1 and VISTA occurs at residue 88, wherein the residue position corresponds to positions 88 of SEQ ID NO: 4. In some cases, the interaction between LRIG1 and VISTA occurs at residue 89, wherein the residue position corresponds to positions 89 of SEQ ID NO: 4. In some cases, the interaction between LRIG1 and VISTA occurs at residue 90, wherein the residue position corresponds to positions 90 of SEQ ID NO: 4. In some cases, VISTA is human VISTA.

In further embodiments, disclosed herein, are methods of promoting B cell or Natural Killer (NK) cell proliferation, comprising contacting a plurality of cells comprising B cells, NK cells, VISTA-expressing cells, and LRIG1-expressing cells with an anti-LRIG1 antibody for a time sufficient to promote proliferation of B cells or NK cells in the plurality of cells. In some embodiments, disclosed herein, are methods of promoting B cell and Natural Killer (NK) cell proliferation, comprising contacting a plurality of cells comprising B cells, NK cells, LRIG1-expressing cells, and VISTA-expressing cells with an anti-LRIG1 antibody for a time sufficient to promote proliferation of B cells and NK cells in the plurality of cells. In some embodiments, disclosed herein, are methods of promoting B cell or Natural Killer (NK) cell proliferation, comprising contacting a plurality of cells comprising one or more cells selected from a group consisting of B cells, NK cells, LRIG1-expressing cells, and VISTA-expressing cells with an anti-LRIG1 antibody for a time sufficient to promote proliferation of B cells or NK cells in the plurality of cells. In some embodiments, disclosed herein, are methods of promoting B cell and Natural Killer (NK) cell proliferation, comprising contacting a plurality of cells comprising one or more cells selected from a group consisting of B cells, NK cells, LRIG1-expressing cells, and VISTA-expressing cells with an anti-LRIG1 antibody for a time sufficient to promote proliferation of B cells and NK cells in the plurality of cells. In some cases, anti-LRIG1 antibody binds to LRIG1 and disrupts an interaction between LRIG1 and VISTA. In some cases, anti-LRIG1 antibody binds to LRIG1 and inhibits an interaction between LRIG1 and VISTA.

In some instances, the LRIG1-expressing cell is a tumor cell or an immune cell. In some cases, the immune cell comprises macrophages, dendritic cells, and IFNγ-producing Th1 cells. In some cases, LRIG1 is expressed in a plurality of cells located within a tumor microenvironment (TME). In some cases, the anti-LRIG1 antibody induces a decrease of tumor cells within the TME. In some cases, the anti-LRIG1 antibody induces a decrease of tumor cells by at least or about 5%, 10%, 15%, 20%, 25%, 30%, 35%, 40%, 45%, 50%, 60%, 70%, 80%, or 90%. In some cases, the anti-LRIG1 antibody induces a decrease of tumor cells in a range of about 5% to about 95%, about 10% to about 90%, about 15% to about 80%, about 20% to about 70%, or about 30% to about 60%. In some cases, the anti-LRIG1 antibody induces a decrease of tumor cells by at least 30%.

In some instances, the plurality of cells further comprises tumor-infiltrating lymphocytes (TILs). In some cases, the plurality of cells further comprises CD3+T lymphocytes, CD4+T helper cells, CD8+ cytotoxic T cells, or a combination thereof. In some cases, the plurality of cells further comprises CD3+T lymphocytes. In some cases, the plurality of cells further comprises CD4+T helper cells. In some cases, the plurality of cells further comprises CD8+ cytotoxic T cells. In some cases, the plurality of cells further comprises CD3+T lymphocytes and CD4+T helper cells. In some cases, the plurality of cells further comprises CD3+T lymphocytes and CD8+ cytotoxic T cells. In some cases, the plurality of cells further comprises CD4+T helper cells, CD8+ cytotoxic T cells. In some cases, the plurality of cells further comprises CD3+T lymphocytes, CD4+T helper cells, and CD8+ cytotoxic T cells.

In some instances, the contacting further induces TIL proliferation. In some cases, the contacting further induces proliferation of CD3+T lymphocytes, CD4+T helper cells, CD8+ cytotoxic T cells, or a combination thereof. In some cases, the contacting further induces proliferation of CD3+T lymphocytes. In some cases, the contacting further induces proliferation of CD4+T helper cells. In some cases, the contacting further induces proliferation of CD8+ cytotoxic T cells. In some cases, the contacting further induces proliferation of CD3+T lymphocytes and CD4+T helper cells. In some cases, the contacting further induces proliferation of CD3+T lymphocytes and CD8+ cytotoxic T cells. In some cases, the contacting further induces proliferation of CD4+T helper cells and CD8+ cytotoxic T cells. In some cases, the contacting further induces proliferation of CD3+T lymphocytes, CD4+T helper cells, and CD8+ cytotoxic T cells.

In some instances, the contacting further comprises an increase in proliferation of M1 macrophages. In some instances, the contacting further comprises a decrease in M2 macrophage population within the TME. In some instances, the contacting further comprises an increase in proliferation of M1 macrophages and a decrease in M2 macrophage population within the TME.

In some instances, the anti-LRIG1 antibody binds to at least one amino acid residue within a LRIG1 region that corresponds to residues 245-260 of SEQ ID NO: 2. In some cases, the anti-LRIG1 antibody binds to at least one amino acid residue within a LRIG1 region that corresponds to residue 245 of SEQ ID NO: 2. In some cases, the anti-LRIG1 antibody binds to at least one amino acid residue within a LRIG1 region that corresponds to residue 246 of SEQ ID NO: 2. In some cases, the anti-LRIG1 antibody binds to at least one amino acid residue within a LRIG1 region that corresponds to residue 247 of SEQ ID NO: 2. In some cases, the anti-LRIG1 antibody binds to at least one amino acid residue within a LRIG1 region that corresponds to residue 248 of SEQ ID NO: 2. In some cases, the anti-LRIG1 antibody binds to at least one amino acid residue within a LRIG1 region that corresponds to residue 249 of SEQ ID NO: 2. In some cases, the anti-LRIG1 antibody binds to at least one amino acid residue within a LRIG1 region that corresponds to residue 250 of SEQ ID NO: 2. In some cases, the anti-LRIG1 antibody binds to at least one amino acid residue within a LRIG1 region that corresponds to residue 251 of SEQ ID NO: 2. In some cases, the anti-LRIG1 antibody binds to at least one amino acid residue within a LRIG1 region that corresponds to residue 252 of SEQ ID NO: 2. In some cases, the anti-LRIG1 antibody binds to at least one amino acid residue within a LRIG1 region that corresponds to residue 253 of SEQ ID NO: 2. In some cases, the anti-LRIG1 antibody binds to at least one amino acid residue within a LRIG1 region that corresponds to residue 254 of SEQ ID NO: 2. In some cases, the anti-LRIG1 antibody binds to at least one amino acid residue within a LRIG1 region that corresponds to residue 255 of SEQ ID NO: 2. In some cases, the anti-LRIG1 antibody binds to at least one amino acid residue within a LRIG1 region that corresponds to residue 256 of SEQ ID NO: 2. In some cases, the anti-LRIG1 antibody binds to at least one amino acid residue within a LRIG1 region that corresponds to residue 257 of SEQ ID NO: 2. In some cases, the anti-LRIG1 antibody binds to at least one amino acid residue within a LRIG1 region that corresponds to residue 258 of SEQ ID NO: 2. In some cases, the anti-LRIG1 antibody binds to at least one amino acid residue within a LRIG1 region that corresponds to residue 259 of SEQ ID NO: 2. In some cases, the anti-LRIG1 antibody binds to at least one amino acid residue within a LRIG1 region that corresponds to residue 260 of SEQ ID NO: 2.

In some cases, the anti-LRIG1 antibody binds to at least one amino acid residue within Peptide 1, Peptide 2, Peptide 3, Peptide 4, Peptide 5, Peptide 6, Peptide 7, Peptide 8, Peptide 9, Peptide 10, Peptide 11, Peptide 12, Peptide 13, Peptide 14, Peptide 15, Peptide 16, Peptide 17, Peptide 18, Peptide 19, Peptide 20, Peptide 21, Peptide 22, Peptide 23, Peptide 24, Peptide 25, Peptide 26, Peptide 27, Peptide 28, Peptide 29, Peptide 30, Peptide 31, Peptide 32, Peptide 33, Peptide 34, Peptide 35, Peptide 36, Peptide 37, Peptide 38, Peptide 39, Peptide 40, Peptide 41, Peptide 42, Peptide 43, Peptide 44, Peptide 45, Peptide 46, Peptide 47, Peptide 48, Peptide 49, Peptide 50, Peptide 51, Peptide 52, Peptide 53, Peptide 54, Peptide 55, Peptide 56, Peptide 57, Peptide 58, Peptide 59, Peptide 60, Peptide 61, Peptide 62, Peptide 63, Peptide 64, Peptide 65, Peptide 66, Peptide 67, Peptide 68, Peptide 69, Peptide 70, Peptide 71, Peptide 72, Peptide 73, Peptide 74, Peptide 75, or Peptide 76. In some cases, the anti-LRIG1 antibody binds to at least one amino acid residue within Peptide 54. In some cases, the anti-LRIG1 antibody binds to at least one amino acid residue within Peptide 61.

In some cases, the anti-LRIG1 antibody binds to at least one amino acid residue within a peptide, wherein the peptide has a sequence as set forth in SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80.

In some instances, the anti-LRIG1 antibody comprises a binding affinity (e.g., kD) to LRIG1 of less than 1 nM, less than 1.2 nM, less than 2 nM, less than 5 nM, less than 10 nM, less than 13.5 nM, less than 15 nM, less than 20 nM, less than 25 nM, or less than 30 nM. In some instances, the anti-LRIG1 antibody comprises a kD of less than 1 nM. In some instances, the anti-LRIG1 antibody comprises a kD of less than 1.2 nM. In some instances, the anti-LRIG1 antibody comprises a kD of less than 2 nM. In some instances, the anti-LRIG1 antibody comprises a kD of less than 5 nM. In some instances, the anti-LRIG1 antibody comprises a kD of less than 10 nM. In some instances, the anti-LRIG1 antibody comprises a kD of less than 13.5 nM. In some instances, the anti-LRIG1 antibody comprises a kD of less than 15 nM. In some instances, the anti-LRIG1 antibody comprises a kD of less than 20 nM. In some instances, the anti-LRIG1 antibody comprises a kD of less than 25 nM. In some instances, the anti-LRIG1 antibody comprises a kD of less than 30 nM.

In some instances, the anti-LRIG1 antibody comprises a humanized antibody. In other instances, the anti-LRIG1 antibody comprises a chimeric antibody. In some cases, the anti-LRIG1 antibody comprises a full-length antibody or a binding fragment thereof. In some cases, the anti-LRIG1 antibody comprises a bispecific antibody or a binding fragment thereof. In some cases, the anti-LRIG1 antibody comprises a monovalent Fab′, a divalent Fab2, a single-chain variable fragment (scFv), a diabody, a minibody, a nanobody, a single-domain antibody (sdAb), or a camelid antibody or binding fragment thereof.

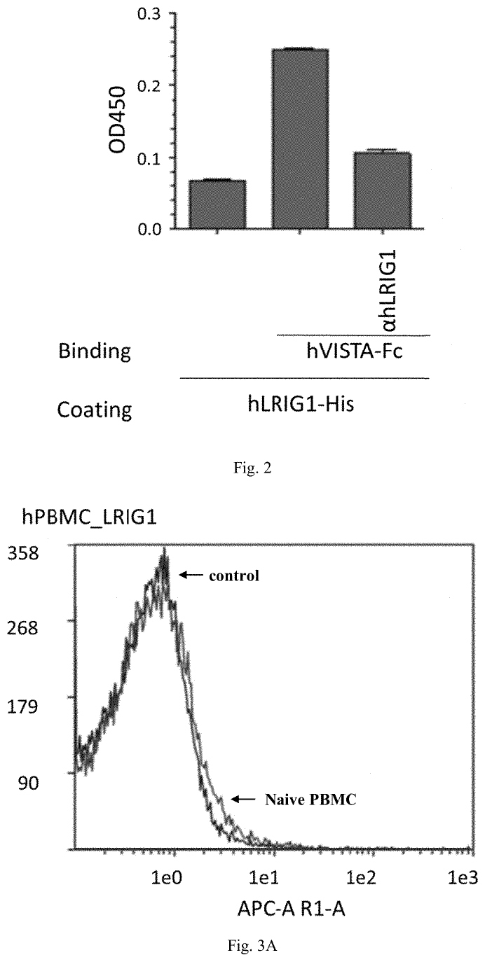

In some instances, the anti-LRIG1 antibody is a bispecific antibody or binding fragment thereof. Exemplary bispecific antibody formats include, but are not limited to, Knobs-into-Holes (KiH), Asymmetric Re-engineering Technology-immunoglobulin (ART-Ig), Triomab quadroma, bispecific monoclonal antibody (BiMAb, BsmAb, BsAb, bsMab, BS-Mab, or Bi-MAb), Azymetric, Bispecific Engagement by Antibodies based on the T-cell receptor (BEAT), Bispecific T-cell Engager (BiTE), Biclonics, Fab-scFv-Fc, Two-in-one/Dual Action Fab (DAF), FinomAb, scFv-Fc-(Fab)-fusion, Dock-aNd-Lock (DNL), Adaptir (previously SCORPION), Tandem diAbody (TandAb), Dual-affinity-ReTargeting (DART), nanobody, triplebody, tandems scFv (taFv), triple heads, tandem dAb/VHH, triple dAb/VHH, or tetravalent dAb/VHH. In some cases, the anti-VISTA antibody, the anti-LRIG1 antibody, or combination thereof is a bispecific antibody or binding fragment thereof comprising a bispecific antibody format illustrated in FIG. 2 of Brinkmann and Kontermann, “The making of bispecific antibodies,” MABS 9(2): 182-212 (2017).

In some embodiments, the anti-LRIG1 antibody is a humanized antibody comprising the complementarity-determining regions (CDRs) illustrated in Table 1 below.

| | SEQUENCE | SEQ ID NO: |
|---|---|---|
| CDR1-H | GYTFTSY | 81 |
| CDR2-H | WIYPGNVNTKYN | 82 |
| CDR3-H | EELGGFAY | 83 |
| CDR1-L | RASQDISNYLS | 84 |
| CDR2-L | YTSILHS | 85 |
| CDR3-L | QQGNTLPRT | 86 |

In some instances, the anti-LRIG1 antibody is a humanized antibody comprising a heavy chain variable region (VH) and a light chain variable region (VL) illustrated in Table 2 below.

| | | SEQ ID |
|---|---|---|
| | SEQUENCE | NO: |
| VH-1 | QVQLVQSGAEVKKPGASVKVSCKASGYTFTSYYLHWVRQAPGQRLEW | 87 |
| | MGWIYPGNVNTKYNQKFQGRVTITADKSASTAYMELSSLRSEDTAVYFC | |
| | AREELGGFAYWGQGTLVTVSS | |
| VH-2 | QVQLVQSGAEVKKPGASVKVSCKASGYTFTSYYLHWVRQAPGQRLEWI | 88 |
| | GWIYPGNVNTKYNEKFQGRVTLTADKSASTAYMELSSLRSEDTAVYFCA | |
| | REELGGFAYWGQGTLVTVSS | |
| VL-1 | DIQMTQSPSSVSASIGDRVTITCRASQDISNYLSWYQQKPGKAPKLLIYYT | 89 |
| | SILHSGVPSRFSGSGSGTDYTLTISSLQPEDFATYFCQQGNTLPRTFGGGTK | |
| | VEIK | |
| VL-2 | DIQMTQSPSSLSASVGNRVTITCRASQDISNYLSWYQQKPGKVPKLLIYYT | 90 |
| | SIHSGVPSRFSGSGSGTDYSLTISSLQPEDVATYFCQQGNTLPRTFGQGTK | |
| | VEIK | |

In some cases, the humanized anti-LRIG1 antibody comprises a VH sequence and a VL sequence as illustrated in Table 3 below.

| | VL-1 | VL-2 |
|---|---|---|
| | (SEQ ID NO: 89) | (SEQ ID NO: 90) |
| VH-1 | mab4 | mab5 |
| (SEQ ID NO: | (SEQ ID NO: 87) + | (SEQ ID NO: 87) + |
| 87) | (SEQ ID NO: 89) | (SEQ ID NO: 90) |
| VH-2 | mab6 | (SEQ ID NO: 88) + |
| (SEQ ID NO: | (SEQ ID NO: 88) + | (SEQ ID NO: 90) |
| 88) | (SEQ ID NO: 89) | |

In some cases, the humanized anti-LRIG1 antibody is mab2, mab4, mab5, or mab6.

In some embodiments, the anti-LRIG1 antibody comprises a framework region selected from IgM, IgG (e.g., IgG1, IgG2, IgG3, or IgG4), IgA, or IgE. In some cases, the anti-LRIG1 antibody comprises an IgM framework. In some cases, the anti-LRIG1 antibody comprises an IgG (e.g., IgG1, IgG2, IgG3, or IgG4) framework. In some cases, the anti-LRIG1 antibody comprises an IgG1 framework. In some cases, the anti-LRIG1 antibody comprises an IgG2 framework. In some cases, the anti-LRIG1 antibody comprises an IgG4 framework.

In some embodiments, the anti-LRIG1 antibody comprises one or more mutations in the framework region, e.g., in the CH1 domain, CH2 domain, CH3 domain, hinge region, or a combination thereof. In some cases, the one or more mutations modulate Fc receptor interactions, e.g., to increase Fc effector functions such as ADCC and/or complement-dependent cytotoxicity (CDC). In some cases, the one or more mutations stabilize the antibody and/or increase the half-life of the antibody. In additional cases, the one or more mutations modulate glycosylation.

### Method of Treatment

In some embodiments, also disclosed herein is a method of administering to a subject in need thereof an anti-LRIG1 antibody described supra. In some instances, the subject is diagnosed with a cancer. In some cases, the cancer is a solid tumor. In other instances, the cancer is a hematologic malignancy. In additional instances, the cancer is a metastatic, a relapsed, or a refractory cancer.

In some instances, the cancer is a solid tumor. In some cases, the cancer is breast cancer. In some cases, the cancer is colorectal cancer. In some cases, the cancer is kidney cancer. In some cases, the cancer is liver cancer. In some cases, the cancer is lung cancer. In some cases, the lung cancer comprises a non-small cell lung cancer (NSCLC) such as lung adenocarcinoma, squamous cell carcinoma, or large cell carcinoma; or small cell lung cancer (SCLC).

In some cases, the cancer is a hematologic malignancy, e.g., a metastatic, relapsed, or refractory hematologic malignancy.

In some instances, the anti-LRIG1 antibody is formulated for systemic administration. In some instances, the anti-LRIG1 antibody is formulated for parenteral administration.

In some embodiments, the anti-LRIG1 antibody is administered to the subject in combination with an additional therapeutic agent. In some instances, the additional therapeutic agent comprises an immunotherapeutic agent. In some instances, the additional therapeutic agent comprises an immune checkpoint modulator. In some instances, the additional therapeutic agent comprises a chemotherapeutic agent, targeted therapeutic agent, hormonal therapeutic agent, or a stem cell-based therapeutic agent.

In some instances, the additional therapeutic agent comprises an immunotherapeutic agent. In some instances, the immunotherapy is an adoptive cell therapy. Exemplary adoptive cell therapies include AFP TCR, MAGE-A10 TCR, or NY-ESO-TCR from Adaptimmune; ACTR087/rituximab from Unum Therapeutics; anti-BCMA CAR-T cell therapy, anti-CD19 “armored” CAR-T cell therapy, JCAR014, JCAR018, JCAR020, JCAR023, JCAR024, or JTCR016 from Juno Therapeutics; JCAR017 from Celgene/Juno Therapeutics; anti-CD19 CAR-T cell therapy from Intrexon; anti-CD19 CAR-T cell therapy, axicabtagene ciloleucel, KITE-718, KITE-439, or NY-ESO-1 T-cell receptor therapy from Kite Pharma; anti-CEA CAR-T therapy from Sorrento Therapeutics; anti-PSMA CAR-T cell therapy from TNK Therapeutics/Sorrento Therapeutics; ATA520 from Atara Biotherapeutics; AU101 and AU105 from Aurora BioPharma; baltaleucel-T (CMD-003) from Cell Medica; bb2121 from bluebird bio; BPX-501, BPX-601, or BPX-701 from Bellicum Pharmaceuticals; BSK01 from Kiromic; IMCgp100 from Immunocore; JTX-2011 from Jounce Therapeutics; LN-144 or LN-145 from Lion Biotechnologies; MB-101 or MB-102 from Mustang Bio; NKR-2 from Celyad; PNK-007 from Celgene; tisagenlecleucel-T from Novartis Pharmaceuticals; or TT12 from Tessa Therapeutics.

In some instances, the immunotherapy is a dendritic cell-based therapy.

In some instances, the immunotherapy comprises a cytokine-based therapy, comprising e.g., an interleukin (IL) such as IL-2, IL-15, or IL-21, interferon (IFN)-α, or granulocyte macrophage colony-stimulating factor (GM-CSF).

In some instances, the immunotherapy comprises an immune checkpoint modulator. Exemplary immune checkpoint modulators include PD-1 modulators such as nivolumab (Opdivo) from Bristol-Myers Squibb, pembrolizumab (Keytruda) from Merck, AGEN 2034 from Agenus, BGB-A317 from BeiGene, B1-754091 from Boehringer-Ingelheim Pharmaceuticals, CBT-501 (genolimzumab) from CBT Pharmaceuticals, INCSHR1210 from Incyte, JNJ-63723283 from Janssen Research & Development, MEDI0680 from MedImmune, MGA 012 from MacroGenics, PDR001 from Novartis Pharmaceuticals, PF-06801591 from Pfizer, REGN2810 (SAR439684) from Regeneron Pharmaceuticals/Sanofi, or TSR-042 from TESARO; CTLA-4 modulators such as ipilimumab (Yervoy), or AGEN 1884 from Agenus; PD-L1 modulators such as durvalumab (Imfinzi) from AstraZeneca, atezolizumab (MPDL3280A) from Genentech, avelumab from EMD Serono/Pfizer, CX-072 from CytomX Therapeutics, FAZ053 from Novartis Pharmaceuticals, KN035 from 3D Medicine/Alphamab, LY3300054 from Eli Lilly, or M7824 (anti-PD-L1/TGFbeta trap) from EMD Serono; LAGS modulators such as BMS-986016 from Bristol-Myers Squibb, IMP701 from Novartis Pharmaceuticals, LAG525 from Novartis Pharmaceuticals, or REGN3767 from Regeneron Pharmaceuticals; OX40 modulators such as BMS-986178 from Bristol-Myers Squibb, GSK3174998 from GlaxoSmithKline, INCAGN1949 from Agenus/Incyte, MEDI0562 from MedImmune, PF-04518600 from Pfizer, or RG7888 from Genentechp; GITR modulators such as GWN323 from Novartis Pharmaceuticals, INCAGN1876 from Agenus/Incyte, MEDI1873 from MedImmune, MK-4166 from Merck, or TRX518 from Leap Therapeutics; KIR modulators such as lirilumab from Bristol-Myers Squibb; or TIM modulators such as MBG453 from Novartis Pharmaceuticals or TSR-022 from Tesaro.

In some instances, the additional therapeutic agent comprises a chemotherapeutic agent. Exemplary chemotherapeutic agents include, but are not limited to, alkylating agents such as cyclophosphamide, mechlorethamine, chlorambucil, melphalan, dacarbazine, or nitrosoureas; anthracyclines such as daunorubicin, doxorubicin, epirubicin, idarubicin, mitoxantrone, or valrubicin; cytoskeletal disruptors such as paclitaxel, docetaxel, abraxane, or taxotere; epothilones; histone deacetylase inhibitors such as vorinostat or romidepsin; topoisomerase I inhibitors such as irinotecan or topotecan; topoisomerase II inhibitors such as etoposide, teniposide, or tafluposide; kinase inhibitors such as bortezomib, erlotinib, gefitinib, imatinib, vemurafenib, or vismodegib; nucleotide analogs and precursor analogs such as azacitidine, azathioprine, capecitabine, cytarabine, doxifluridine, fluorouracil, gemcitabine, hydrozyurea, mercaptopurine, methotrexate, or tioguanine; platinum-based agents such as carboplatin, cisplatin, or oxaliplatin; retinoids such as tretinoin, alitretinoin, or bexarotene; or vinca alkaloids and derivatives such as vinblastine, vincristine, vindesine, or vinorelbine.

In some instances, the additional therapeutic agent comprises a hormone-based therapeutic agent. Exemplary hormone-based therapeutic agents include, but are not limited to, aromatase inhibitors such as letrozole, anastrozole, exemestane, or aminoglutethimide; gonadotropin-releasing hormone (GnRH) analogues such as leuprorelin or goserelin; selective estrogen receptor modulators (SERMs) such as tamoxifen, raloxifene, toremifene, or fulvestrant; antiandrogens such as flutamide or bicalutamide; progestogens such as megestrol acetate or medroxyprogesterone acetate; androgens such as fluoxymesterone; estrogens such as estrogen diethylstilbestrol (DES), Estrace, or polyestradiol phosphate; or somatostatin analogs such as octreotide.

In some instances, the additional therapeutic agent is a first-line therapeutic agent.

In some embodiments, the anti-LRIG1 antibody and the additional therapeutic agent are administered simultaneously. In some instances, the anti-LRIG1 antibody and the additional therapeutic agent are administered sequentially. In such instances, the anti-LRIG1 antibody is administered to the subject prior to administering the additional therapeutic agent. In other instances, the anti-LRIG1 antibody is administered to the subject after the additional therapeutic agent is administered.

In some cases, the additional therapeutic agent and the anti-LRIG1 antibody are formulated as separate dosage.

In some instances, the subject has undergone surgery. In some cases, the anti-LRIG1 antibody and optionally the additional therapeutic agent are administered to the subject prior to surgery. In some instances, the anti-LRIG1 antibody and optionally the additional therapeutic agent are administered to the subject after surgery.

In some instances, the subject has undergone radiation. In some instances, the anti-LRIG1 antibody and optionally the additional therapeutic agent are administered to the subject during or after radiation treatment. In some cases, the anti-LRIG1 antibody and optionally the additional therapeutic agent are administered to the subject prior to undergoing radiation.

In some instances, the subject is a human

### Antibody Production

In some embodiments, anti-LRIG1 antibodies are raised by standard protocol by injecting a production animal with an antigenic composition. See, e.g., Harlow and Lane, Antibodies: A Laboratory Manual, Cold Spring Harbor Laboratory, 1988. When utilizing an entire protein, or a larger section of the protein, antibodies may be raised by immunizing the production animal with the protein and a suitable adjuvant (e.g., Freund's, Freund's complete, oil-in-water emulsions, etc.). When a smaller peptide is utilized, it is advantageous to conjugate the peptide with a larger molecule to make an immunostimulatory conjugate. Commonly utilized conjugate proteins that are commercially available for such use include bovine serum albumin (BSA) and keyhole limpet hemocyanin (KLH). In order to raise antibodies to particular epitopes, peptides derived from the full sequence may be utilized. Alternatively, in order to generate antibodies to relatively short peptide portions of the protein target, a superior immune response may be elicited if the polypeptide is joined to a carrier protein, such as ovalbumin, BSA or KLH.

Polyclonal or monoclonal anti-LRIG1 antibodies can be produced from animals which have been genetically altered to produce human immunoglobulins. A transgenic animal can be produced by initially producing a “knock-out” animal which does not produce the animal's natural antibodies, and stably transforming the animal with a human antibody locus (e.g., by the use of a human artificial chromosome). In such cases, only human antibodies are then made by the animal. Techniques for generating such animals, and deriving antibodies therefrom, are described in U.S. Pat. Nos. 6,162,963 and 6,150,584, incorporated fully herein by reference. Such antibodies can be referred to as human xenogenic antibodies.

Alternatively, anti-LRIG1 antibodies can be produced from phage libraries containing human variable regions. See U.S. Pat. No. 6,174,708, incorporated fully herein by reference.

In some aspects of any of the embodiments disclosed herein, an anti-LRIG1 antibody is produced by a hybridoma.

For monoclonal anti-LRIG1 antibodies, hybridomas may be formed by isolating the stimulated immune cells, such as those from the spleen of the inoculated animal. These cells can then be fused to immortalized cells, such as myeloma cells or transformed cells, which are capable of replicating indefinitely in cell culture, thereby producing an immortal, immunoglobulin-secreting cell line. The immortal cell line utilized can be selected to be deficient in enzymes necessary for the utilization of certain nutrients. Many such cell lines (such as myelomas) are known to those skilled in the art, and include, for example: thymidine kinase (TK) or hypoxanthine-guanine phosphoriboxyl transferase (HGPRT). These deficiencies allow selection for fused cells according to their ability to grow on, for example, hypoxanthine aminopterinthymidine medium (HAT).

In addition, the anti-LRIG1 antibody may be produced by genetic engineering.

Anti-LRIG1 antibodies disclosed herein can have a reduced propensity to induce an undesired immune response in humans, for example, anaphylactic shock, and can also exhibit a reduced propensity for priming an immune response which would prevent repeated dosage with an antibody therapeutic or imaging agent (e.g., the human-anti-murine-antibody “HAMA” response). Such anti-LRIG1 antibodies include, but are not limited to, humanized, chimeric, or xenogenic human anti-LRIG1 antibodies.

Chimeric anti-LRIG1 antibodies can be made, for example, by recombinant means by combining the murine variable light and heavy chain regions (VK and VH), obtained from a murine (or other animal-derived) hybridoma clone, with the human constant light and heavy chain regions, in order to produce an antibody with predominantly human domains. The production of such chimeric antibodies is well known in the art, and may be achieved by standard means (as described, e.g., in U.S. Pat. No. 5,624,659, incorporated fully herein by reference).

The term “humanized” as applies to a non-human (e.g. rodent or primate) antibodies are hybrid immunoglobulins, immunoglobulin chains or fragments thereof which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, rabbit or primate having the desired specificity, affinity and capacity. In some instances, Fv framework region (FR) residues of the human immunoglobulin are replaced by corresponding non-human residues. Furthermore, the humanized antibody may comprise residues which are found neither in the recipient antibody nor in the imported CDR or framework sequences. These modifications are made to further refine and optimize antibody performance and minimize immunogenicity when introduced into a human body. In some examples, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody may also comprise at least a portion of an immunoglobulin constant region (Fc), typically that of a human immunoglobulin.

Humanized antibodies can be engineered to contain human-like immunoglobulin domains, and incorporate only the complementarity-determining regions of the animal-derived antibody. This can be accomplished by carefully examining the sequence of the hyper-variable loops of the variable regions of a monoclonal antigen binding unit or monoclonal antibody, and fitting them to the structure of a human antigen binding unit or human antibody chains. See, e.g., U.S. Pat. No. 6,187,287, incorporated fully herein by reference.

Methods for humanizing non-human antibodies are well known in the art. “Humanized” antibodies are antibodies in which at least part of the sequence has been altered from its initial form to render it more like human immunoglobulins. In some versions, the heavy (H) chain and light (L) chain constant (C) regions are replaced with human sequence. This can be a fusion polypeptide comprising a variable (V) region and a heterologous immunoglobulin C region. In some versions, the complementarity determining regions (CDRs) comprise non-human antibody sequences, while the V framework regions have also been converted to human sequences. See, for example, EP 0329400. In some versions, V regions are humanized by designing consensus sequences of human and mouse V regions, and converting residues outside the CDRs that are different between the consensus sequences.

In principle, a framework sequence from a humanized antibody can serve as the template for CDR grafting; however, it has been demonstrated that straight CDR replacement into such a framework can lead to significant loss of binding affinity to the antigen. Glaser et al. (1992) J. Immunol. 149:2606; Tempest et al. (1992) Biotechnology 9:266; and Shalaby et al. (1992) J. Exp. Med. 17:217. The more homologous a human antibody (HuAb) is to the original murine antibody (muAb), the less likely that the human framework will introduce distortions into the murine CDRs that could reduce affinity. Based on a sequence homology search against an antibody sequence database, the HuAb IC4 provides good framework homology to muM4TS.22, although other highly homologous HuAbs would be suitable as well, especially kappa L chains from human subgroup I or H chains from human subgroup III. Kabat et al. (1987). Various computer programs such as ENCAD (Levitt et al. (1983) J. Mol. Biol. 168:595) are available to predict the ideal sequence for the V region. The invention thus encompasses HuAbs with different variable (V) regions. It is within the skill of one in the art to determine suitable V region sequences and to optimize these sequences. Methods for obtaining antibodies with reduced immunogenicity are also described in U.S. Pat. No. 5,270,202 and EP 699,755.

Humanized antibodies can be prepared by a process of analysis of the parental sequences and various conceptual humanized products using three dimensional models of the parental and humanized sequences. Three dimensional immunoglobulin models are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved.

A process for humanization of subject antigen binding units can be as follows. The best-fit germline acceptor heavy and light chain variable regions are selected based on homology, canonical structure and physical properties of the human antibody germlines for grafting. Computer modeling of mVH/VL versus grafted hVH/VL is performed and prototype humanized antibody sequence is generated. If modeling indicated a need for framework back-mutations, second variant with indicated FW changes is generated. DNA fragments encoding the selected germline frameworks and murine CDRs are synthesized. The synthesized DNA fragments are subcloned into IgG expression vectors and sequences are confirmed by DNA sequencing. The humanized antibodies are expressed in cells, such as 293F and the proteins are tested, for example in MDM phagocytosis assays and antigen binding assays. The humanized antigen binding units are compared with parental antigen binding units in antigen binding affinity, for example, by FACS on cells expressing the target antigen. If the affinity is greater than 2-fold lower than parental antigen binding unit, a second round of humanized variants can be generated and tested as described above.

As noted above, an anti-LRIG1 antibody can be either “monovalent” or “multivalent.” Whereas the former has one binding site per antigen-binding unit, the latter contains multiple binding sites capable of binding to more than one antigen of the same or different kind. Depending on the number of binding sites, antigen binding units may be bivalent (having two antigen-binding sites), trivalent (having three antigen-binding sites), tetravalent (having four antigen-binding sites), and so on.

Multivalent anti-LRIG1 antibodies can be further classified on the basis of their binding specificities. A “monospecific” anti-LRIG1 antibody is a molecule capable of binding to one or more antigens of the same kind. A “multispecific” anti-LRIG1 antibody is a molecule having binding specificities for at least two different antigens. While such molecules normally will only bind two distinct antigens (i.e. bispecific anti-LRIG1 antibodies), antibodies with additional specificities such as trispecific antibodies are encompassed by this expression when used herein. This disclosure further provides multispecific anti-LRIG1 antibodies. Multispecific anti-LRIG1 antibodies are multivalent molecules capable of binding to at least two distinct antigens, e.g., bispecific and trispecific molecules exhibiting binding specificities to two and three distinct antigens, respectively.

### Polynucleotides and Vectors

In some embodiments, the present disclosure provides isolated nucleic acids encoding any of the anti-LRIG1 antibodies disclosed herein. In another embodiment, the present disclosure provides vectors comprising a nucleic acid sequence encoding any anti-LRIG1 antibody disclosed herein. In some embodiments, this invention provides isolated nucleic acids that encode a light-chain CDR and a heavy-chain CDR of an anti-LRIG1 antibody disclosed herein.

The subject anti-LRIG1 antibodies can be prepared by recombinant DNA technology, synthetic chemistry techniques, or a combination thereof. For instance, sequences encoding the desired components of the anti-LRIG1 antibodies, including light chain CDRs and heavy chain CDRs are typically assembled cloned into an expression vector using standard molecular techniques know in the art. These sequences may be assembled from other vectors encoding the desired protein sequence, from PCR-generated fragments using respective template nucleic acids, or by assembly of synthetic oligonucleotides encoding the desired sequences. Expression systems can be created by transfecting a suitable cell with an expressing vector which comprises an anti-LRIG1 antibody of interest.

Nucleotide sequences corresponding to various regions of light or heavy chains of an existing antibody can be readily obtained and sequenced using convention techniques including but not limited to hybridization, PCR, and DNA sequencing. Hybridoma cells that produce monoclonal antibodies serve as a preferred source of antibody nucleotide sequences. A vast number of hybridoma cells producing an array of monoclonal antibodies may be obtained from public or private repositories. The largest depository agent is American Type Culture Collection (atcc.org), which offers a diverse collection of well-characterized hybridoma cell lines. Alternatively, antibody nucleotides can be obtained from immunized or non-immunized rodents or humans, and form organs such as spleen and peripheral blood lymphocytes. Specific techniques applicable for extracting and synthesizing antibody nucleotides are described in Orlandi et al. (1989) Proc. Natl. Acad. Sci. U.S.A 86: 3833-3837; Larrick et al. (1989) Biochem. Biophys. Res. Commun. 160:1250-1255; Sastry et al. (1989) Proc. Natl. Acad. Sci., U.S.A. 86: 5728-5732; and U.S. Pat. No. 5,969,108.

Polynucleotides encoding anti-LRIG1 antibodies can also be modified, for example, by substituting the coding sequence for human heavy and light chain constant regions in place of the homologous non-human sequences. In that manner, chimeric antibodies are prepared that retain the binding specificity of the original anti-LRIG1 antibody.

### Host Cells

In some embodiments, the present disclosure provides host cells expressing any one of the anti-LRIG1 antibodies disclosed herein. A subject host cell typically comprises a nucleic acid encoding any one of the anti-LRIG1 antibodies disclosed herein.

The invention provides host cells transfected with the polynucleotides, vectors, or a library of the vectors described above. The vectors can be introduced into a suitable prokaryotic or eukaryotic cell by any of a number of appropriate means, including electroporation, microprojectile bombardment; lipofection, infection (where the vector is coupled to an infectious agent), transfection employing calcium chloride, rubidium chloride, calcium phosphate, DEAE-dextran, or other substances. The choice of the means for introducing vectors will often depend on features of the host cell.

For most animal cells, any of the above-mentioned methods is suitable for vector delivery. Preferred animal cells are vertebrate cells, preferably mammalian cells, capable of expressing exogenously introduced gene products in large quantity, e.g. at the milligram level. Non-limiting examples of preferred cells are NIH3T3 cells, COS, HeLa, and CHO cells.

Once introduced into a suitable host cell, expression of the anti-LRIG1 antibodies can be determined using any nucleic acid or protein assay known in the art. For example, the presence of transcribed mRNA of light chain CDRs or heavy chain CDRs, or the anti-LRIG1 antibody can be detected and/or quantified by conventional hybridization assays (e.g. Northern blot analysis), amplification procedures (e.g. RT-PCR), SAGE (U.S. Pat. No. 5,695,937), and array-based technologies (see e.g. U.S. Pat. Nos. 5,405,783, 5,412,087 and 5,445,934), using probes complementary to any region of a polynucleotide that encodes the anti-LRIG1 antibody.

Expression of the vector can also be determined by examining the expressed anti-LRIG1 antibody. A variety of techniques are available in the art for protein analysis. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunoflourescent assays, and SDS-PAGE.

### Payload

In some embodiments, an anti-LRIG1 antibody further comprises a payload. In some cases, the payload comprises a small molecule, a protein or functional fragment thereof, a peptide, or a nucleic acid polymer.

In some cases, the number of payloads conjugated to the anti-LRIG1 antibody (e.g., the drug-to-antibody ratio or DAR) is about 1:1, one payload to one anti-LRIG1 antibody. In some cases, the ratio of the payloads to the anti-LRIG1 antibody is about 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1. In some cases, the ratio of the payloads to the anti-LRIG1 antibody is about 2:1. In some cases, the ratio of the payloads to the anti-LRIG1 antibody is about 3:1. In some cases, the ratio of the payloads to the anti-LRIG1 antibody is about 4:1. In some cases, the ratio of the payloads to the anti-LRIG1 antibody is about 6:1. In some cases, the ratio of the payloads to the anti-LRIG1 antibody is about 8:1. In some cases, the ratio of the payloads to the anti-LRIG1 antibody is about 12:1.

In some embodiment, the payload is a small molecule. In some instances, the small molecule is a cytotoxic payload. Exemplary cytotoxic payloads include, but are not limited to, microtubule disrupting agents, DNA modifying agents, or Akt inhibitors.

In some embodiments, the payload comprises a microtubule disrupting agent. Exemplary microtubule disrupting agents include, but are not limited to, 2-methoxyestradiol, auristatin, chalcones, colchicine, combretastatin, cryptophycin, dictyostatin, discodermolide, dolastain, eleutherobin, epothilone, halichondrin, laulimalide, maytansine, noscapinoid, paclitaxel, peloruside, phomopsin, podophyllotoxin, rhizoxin, spongistatin, taxane, tubulysin, vinca alkaloid, vinorelbine, or derivatives or analogs thereof.

In some embodiments, the maytansine is a maytansinoid. In some embodiments, the maytansinoid is DM1, DM4, or ansamitocin. In some embodiments, the maytansinoid is DM1. In some embodiments, the maytansinoid is DM4. In some embodiments, the maytansinoid is ansamitocin. In some embodiments, the maytansinoid is a maytansionid derivative or analog such as described in U.S. Pat. Nos. 5,208,020, 5,416,064, 7,276,497, and 6,716,821 or U.S. Publication Nos. 2013029900 and US20130323268.

In some embodiments, the payload is a dolastatin, or a derivative or analog thereof. In some embodiments, the dolastatin is dolastatin 10 or dolastatin 15, or derivatives or analogs thereof. In some embodiments, the dolastatin 10 analog is auristatin, soblidotin, symplostatin 1, or symplostatin 3. In some embodiments, the dolastatin 15 analog is cemadotin or tasidotin.

In some embodiments, the dolastatin 10 analog is auristatin or an auristatin derivative. In some embodiments, the auristatin or auristatin derivative is auristatin E (AE), auristatin F (AF), auristatin E5-benzoylvaleric acid ester (AEVB), monomethyl auristatin E (MMAE), monomethyl auristatin F (MMAF), or monomethyl auristatin D (MMAD), auristatin PE, or auristatin PYE. In some embodiments, the auristatin derivative is monomethyl auristatin E (MMAE). In some embodiments, the auristatin derivative is monomethyl auristatin F (MMAF). In some embodiments, the auristatin is an auristatin derivative or analog such as described in U.S. Pat. Nos. 6,884,869, 7,659,241, 7,498,298, 7,964,566, 7,750,116, 8,288,352, 8,703,714, and 8,871,720.

In some embodiments, the payload comprises a DNA modifying agent. In some embodiments, the DNA modifying agent comprises DNA cleavers, DNA intercalators, DNA transcription inhibitors, or DNA cross-linkers. In some instances, the DNA cleaver comprises bleomycine A2, calicheamicin, or derivatives or analogs thereof. In some instances, the DNA intercalator comprises doxorubicin, epirubicin, PNU-159682, duocarmycin, pyrrolobenzodiazepine, oligomycin C, daunorubicin, valrubicin, topotecan, or derivatives or analogs thereof. In some instances, the DNA transcription inhibitor comprises dactinomycin. In some instances, the DNA cross-linker comprises mitomycin C.

In some embodiments, the DNA modifying agent comprises amsacrine, anthracycline, camptothecin, doxorubicin, duocarmycin, enediyne, etoposide, indolinobenzodiazepine, netropsin, teniposide, or derivatives or analogs thereof.

In some embodiments, the anthracycline is doxorubicin, daunorubicin, epirubicin, idarubicin, mitomycin-C, dactinomycin, mithramycin, nemorubicin, pixantrone, sabarubicin, or valrubicin.

In some embodiments, the analog of camptothecin is topotecan, irinotecan, silatecan, cositecan, exatecan, lurtotecan, gimatecan, belotecan, rubitecan, or SN-38.

In some embodiments, the duocarmycin is duocarmycin A, duocarmycin B1, duocarmycin B2, duocarmycin C1, duocarmycin C2, duocarmycin D, duocarmycin SA, or CC-1065. In some embodiments, the enediyne is a calicheamicin, esperamicin, or dynemicin A.

In some embodiments, the pyrrolobenzodiazepine is anthramycin, abbeymycin, chicamycin, DC-81, mazethramycin, neothramycins A, neothramycin B, porothramycin, prothracarcin, sibanomicin (DC-102), sibiromycin, or tomaymycin. In some embodiments, the pyrrolobenzodiazepine is a tomaymycin derivative, such as described in U.S. Pat. Nos. 8,404,678 and 8,163,736. In some embodiments, the pyrrolobenzodiazepine is such as described in U.S. Pat. Nos. 8,426,402, 8,802,667, 8,809,320, 6,562,806, 6,608,192, 7,704,924, 7,067,511, 7,612,062, 7,244,724, 7,528,126, 7,049,311, 8,633,185, 8,501,934, and 8,697,688 and U.S. Publication No. US20140294868.

In some embodiments, the pyrrolobenzodiazepine is a pyrrolobenzodiazepine dimer. In some embodiments, the PBD dimer is a symmetric dimer. Examples of symmetric PBD dimers include, but are not limited to, SJG-136 (SG-2000), ZC-423 (SG2285), SJG-720, SJG-738, ZC-207 (SG2202), and DSB-120 (Table 2). In some embodiments, the PBD dimer is an unsymmetrical dimer. Examples of unsymmetrical PBD dimers include, but are not limited to, SJG-136 derivatives such as described in U.S. Pat. Nos. 8,697,688 and 9,242,013 and U.S. Publication No. 20140286970.

In some embodiments, the payload comprises an Akt inhibitor. In some cases, the Akt inhibitor comprises ipatasertib (GDC-0068) or derivatives thereof.

In some embodiments, the payload comprises a polymerase inhibitor, including, but not limited to polymerase II inhibitors such as α-amanitin, and poly(ADP-ribose) polymerase (PARP) inhibitors. Exemplary PARP inhibitors include, but are not limited to Iniparib (BSI 201), Talazoparib (BMN-673), Olaparib (AZD-2281), Olaparib, Rucaparib (AG014699, PF-01367338), Veliparib (ABT-888), CEP 9722, MK 4827, BGB-290, or 3-aminobenzamide.

In some embodiments, the payload comprises a detectable moiety. Exemplary detectable moieties include fluorescent dyes; enzymes; substrates; chemiluminescent moieties; specific binding moieties such as streptavidin, avidin, or biotin; or radioisotopes.

In some embodiments, the payload comprises an immunomodulatory agent. Useful immunomodulatory agents include anti-hormones that block hormone action on tumors and immunosuppressive agents that suppress cytokine production, down-regulate self-antigen expression, or mask MHC antigens. Representative anti-hormones include anti-estrogens including, for example, tamoxifen, raloxifene, aromatase inhibiting 4(5)-imidazoles, 4-hydroxytamoxifen, trioxifene, keoxifene, LY 117018, onapnstone, and toremifene; and anti-androgens such as flutamide, nilutamide, bicalutamide, leuprolide, and goserelin; and anti-adrenal agents. Illustrative immunosuppressive agents include, but are not limited to 2-amino-6-aryl-5-substituted pyrimidines, azathioprine, cyclophosphamide, bromocryptine, danazol, dapsone, glutaraldehyde, anti-idiotypic antibodies for MHC antigens and MHC fragments, cyclosporin A, steroids such as glucocorticosteroids, streptokinase, or rapamycin.

In some embodiments, the payload comprises an immune modulator. Exemplary immune modulators include, but are not limited to, gancyclovier, etanercept, tacrolimus, sirolimus, voclosporin, cyclosporine, rapamycin, cyclophosphamide, azathioprine, mycophenolgate mofetil, methotrextrate, glucocorticoid and its analogs, xanthines, stem cell growth factors, lymphotoxins, hematopoietic factors, tumor necrosis factor (TNF) (e.g., TNFα), interleukins (e.g., interleukin-1 (IL-1), IL-2, IL-3, IL-6, IL-10, IL-12, IL-18, and IL-21), colony stimulating factors (e.g., granulocyte-colony stimulating factor (G-CSF) and granulocyte macrophage-colony stimulating factor (GM-CSF)), interferons (e.g., interferons-alpha, interferon-beta, interferon-gamma), the stem cell growth factor designated “S1 factor,” erythropoietin and thrombopoietin, or a combination thereof.

In some embodiments, the payload comprises an immunotoxin Immunotoxins include, but are not limited to, ricin, radionuclides, pokeweed antiviral protein, Pseudomonas exotoxin A, diphtheria toxin, ricin A chain, fungal toxins such as restrictocin and phospholipase enzymes. See, generally, “Chimeric Toxins,” Olsnes and Pihl, Pharmac. Ther. 15:355-381 (1981); and “Monoclonal Antibodies for Cancer Detection and Therapy,” eds. Baldwin and Byers, pp. 159-179, 224-266, Academic Press (1985).

In some instances, the payload comprises a nucleic acid polymer. In such instances, the nucleic acid polymer comprises short interfering nucleic acid (siNA), short interfering RNA (siRNA), double-stranded RNA (dsRNA), micro-RNA (miRNA), short hairpin RNA (shRNA), an antisense oligonucleotide. In other instances, the nucleic acid polymer comprises an mRNA, encoding, e.g., a cytotoxic protein or peptide or an apoptotic triggering protein or peptide. Exemplary cytotoxic proteins or peptides include a bacterial cytotoxin such as an alpha-pore forming toxin (e.g., cytolysin A from E. coli), a beta-pore-forming toxin (e.g., α-Hemolysin, PVL—panton Valentine leukocidin, aerolysin, clostridial Epsilon-toxin, Clostridium perfringens enterotoxin), binary toxins (anthrax toxin, edema toxin, C. botulinum C2 toxin, C spirofome toxin, C. perfringens iota toxin, C. difficile cyto-lethal toxins (A and B)), prion, parasporin, a cholesterol-dependent cytolysins (e.g., pneumolysin), a small pore-forming toxin (e.g., Gramicidin A), a cyanotoxin (e.g., microcystins, nodularins), a hemotoxin, a neurotoxin (e.g., botulinum neurotoxin), a cytotoxin, cholera toxin, diphtheria toxin, Pseudomonas exotoxin A, tetanus toxin, or an immunotoxin (idarubicin, ricin A, CRM9, Pokeweed antiviral protein, DT). Exemplary apoptotic triggering proteins or peptides include apoptotic protease activating factor-1 (Apaf-1), cytochrome-c, caspase initiator proteins (CASP2, CASP8, CASP9, CASP10), apoptosis inducing factor (AIF), p53, p73, p63, Bcl-2, Bax, granzyme B, poly-ADP ribose polymerase (PARP), and P 21-activated kinase 2 (PAK2). In additional instances, the nucleic acid polymer comprises a nucleic acid decoy. In some instances, the nucleic acid decoy is a mimic of protein-binding nucleic acids such as RNA-based protein-binding mimics Exemplary nucleic acid decoys include transactivating region (TAR) decoy and Rev response element (RRE) decoy.

In some cases, the payload is an aptamer. Aptamers are small oligonucleotide or peptide molecules that bind to specific target molecules. Exemplary nucleic acid aptamers include DNA aptamers, RNA aptamers, or XNA aptamers which are RNA and/or DNA aptamers comprising one or more unnatural nucleotides. Exemplary nucleic acid aptamers include ARC19499 (Archemix Corp.), REG1 (Regado Biosciences), and ARC1905 (Ophthotech).

Nucleic acids in accordance with the embodiments described herein optionally include naturally occurring nucleic acids, or one or more nucleotide analogs or have a structure that otherwise differs from that of a naturally occurring nucleic acid. For example, 2′-modifications include halo, alkoxy, and allyloxy groups. In some embodiments, the 2′-OH group is replaced by a group selected from H, OR, R, halo, SH, SR, NH₂, NHR, NR₂ or CN, wherein R is C₁-C₆ alkyl, alkenyl, or alkynyl, and halo is F, Cl, Br, or I. Examples of modified linkages include phosphorothioate and 5′-N-phosphoramidite linkages.

Nucleic acids having a variety of different nucleotide analogs, modified backbones, or non-naturally occurring internucleoside linkages are utilized in accordance with the embodiments described herein. In some cases, nucleic acids include natural nucleosides (i.e., adenosine, thymidine, guanosine, cytidine, uridine, deoxyadenosine, deoxythymidine, deoxyguanosine, and deoxycytidine) or modified nucleosides. Examples of modified nucleotides include base modified nucleoside (e.g., aracytidine, inosine, isoguanosine, nebularine, pseudouridine, 2,6-diaminopurine, 2-aminopurine, 2-thiothymidine, 3-deaza-5-azacytidine, 2′-deoxyuridine, 3-nitorpyrrole, 4-methylindole, 4-thiouridine, 4-thiothymidine, 2-aminoadenosine, 2-thiothymidine, 2-thiouridine, 5-bromocytidine, 5-iodouridine, inosine, 6-azauridine, 6-chloropurine, 7-deazaadenosine, 7-deazaguanosine, 8-azaadenosine, 8-azidoadenosine, benzimidazole, M1-methyladenosine, pyrrolo-pyrimidine, 2-amino-6-chloropurine, 3-methyl adenosine, 5-propynylcytidine, 5-propynyluridine, 5-bromouridine, 5-fluorouridine, 5-methylcytidine, 7-deazaadenosine, 7-deazaguanosine, 8-oxoadenosine, 8-oxoguanosine, O(6)-methylguanine, and 2-thiocytidine), chemically or biologically modified bases (e.g., methylated bases), modified sugars (e.g., 2′-fluororibose, 2′-aminoribose, 2′-azidoribose, 2′-O-methylribose, L-enantiomeric nucleosides arabinose, and hexose), modified phosphate groups (e.g., phosphorothioates and 5′-N-phosphoramidite linkages), and combinations thereof. Natural and modified nucleotide monomers for the chemical synthesis of nucleic acids are readily available. In some cases, nucleic acids comprising such modifications display improved properties relative to nucleic acids consisting only of naturally occurring nucleotides. In some embodiments, nucleic acid modifications described herein are utilized to reduce and/or prevent digestion by nucleases (e.g. exonucleases, endonucleases, etc.). For example, the structure of a nucleic acid may be stabilized by including nucleotide analogs at the 3′ end of one or both strands order to reduce digestion.

Different nucleotide modifications and/or backbone structures may exist at various positions in the nucleic acid. Such modification include morpholinos, peptide nucleic acids (PNAs), methylphosphonate nucleotides, thiolphosphonate nucleotides, 2′-fluoro N3-P5′-phosphoramidites, 1′, 5′-anhydrohexitol nucleic acids (HNAs), or a combination thereof.

### Conjugation Chemistry

In some instances, the payload is conjugated to an anti-LRIG1 antibody described herein by a native ligation. In some instances, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology.,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some instances, the conjugation is as described in U.S. Pat. No. 8,936,910.

In some instances, the payload is conjugated to an anti-LRIG1 antibody described herein by a site-directed method utilizing a “traceless” coupling technology (Philochem). In some instances, the “traceless” coupling technology utilizes an N-terminal 1,2-aminothiol group on the binding moiety which is then conjugate with a polynucleic acid molecule containing an aldehyde group. (see Casi et al., “Site-specific traceless coupling of potent cytotoxic drugs to recombinant antibodies for pharmacodelivery,” JACS 134(13): 5887-5892 (2012))

In some instances, the payload is conjugated to an anti-LRIG1 antibody described herein by a site-directed method utilizing an unnatural amino acid incorporated into the binding moiety. In some instances, the unnatural amino acid comprises p-acetylphenylalanine (pAcPhe). In some instances, the keto group of pAcPhe is selectively coupled to an alkoxy-amine derivatived conjugating moiety to form an oxime bond. (see Axup et al., “Synthesis of site-specific antibody-drug conjugates using unnatural amino acids,” PNAS 109(40): 16101-16106 (2012)).

In some instances, the payload is conjugated to an anti-LRIG1 antibody described herein by a site-directed method utilizing an enzyme-catalyzed process. In some instances, the site-directed method utilizes SMARTag™ technology (Redwood). In some instances, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized polynucleic acid molecule via hydrazino-Pictet-Spengler (HIPS) ligation. (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013)).

In some instances, the enzyme-catalyzed process comprises microbial transglutaminase (mTG). In some cases, the payload is conjugated to the anti-LRIG1 antibody utilizing a microbial transglutaminze catalyzed process. In some instances, mTG catalyzes the formation of a covalent bond between the amide side chain of a glutamine within the recognition sequence and a primary amine of a functionalized polynucleic acid molecule. In some instances, mTG is produced from Streptomyces mobarensis. (see Strop et al., “Location matters: site of conjugation modulates stability and pharmacokinetics of antibody drug conjugates,” Chemistry and Biology 20(2) 161-167 (2013)).

In some instances, the payload is conjugated to an anti-LRIG1 antibody by a method as described in PCT Publication No. WO2014/140317, which utilizes a sequence-specific transpeptidase.

In some instances, the payload is conjugated to an anti-LRIG1 antibody described herein by a method as described in U.S. Patent Publication Nos. 2015/0105539 and 2015/0105540.

### Linker

In some instances, a linker described above comprises a natural or synthetic polymer, consisting of long chains of branched or unbranched monomers, and/or cross-linked network of monomers in two or three dimensions. In some instances, the linker includes a polysaccharide, lignin, rubber, or polyalkylen oxide (e.g., polyethylene glycol).

In some instances, the linker includes, but is not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethylenterephthalat (PET, PETG), polyethylene terephthalate (PETE), polytetramethylene glycol (PTG), or polyurethane as well as mixtures thereof. As used herein, a mixture refers to the use of different polymers within the same compound as well as in reference to block copolymers. In some cases, block copolymers are polymers wherein at least one section of a polymer is build up from monomers of another polymer. In some instances, the linker comprises polyalkylene oxide. In some instances, the linker comprises PEG. In some instances, the linker comprises polyethylene imide (PEI) or hydroxy ethyl starch (HES).

In some cases, the polyalkylene oxide (e.g., PEG) is a polydispers or monodispers compound. In some instances, polydispers material comprises disperse distribution of different molecular weight of the material, characterized by mean weight (weight average) size and dispersity. In some instances, the monodisperse PEG comprises one size of molecules. In some embodiments, the linker is poly- or monodispersed polyalkylene oxide (e.g., PEG) and the indicated molecular weight represents an average of the molecular weight of the polyalkylene oxide, e.g., PEG, molecules.

In some embodiments, the linker comprises a polyalkylene oxide (e.g., PEG) and the molecular weight of the polyalkylene oxide (e.g., PEG) is about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da.

In some embodiments, the polyalkylene oxide (e.g., PEG) is a discrete PEG, in which the discrete PEG is a polymeric PEG comprising more than one repeating ethylene oxide units. In some instances, a discrete PEG (dPEG) comprises from 2 to 60, from 2 to 50, or from 2 to 48 repeating ethylene oxide units. In some instances, a dPEG comprises about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units. In some instances, a dPEG comprises about 2 or more repeating ethylene oxide units. In some cases, a dPEG is synthesized as a single molecular weight compound from pure (e.g., about 95%, 98%, 99%, or 99.5%) staring material in a step-wise fashion. In some cases, a dPEG has a specific molecular weight, rather than an average molecular weight. In some cases, a dPEG described herein is a dPEG from Quanta Biodesign, LMD.

In some instances, the linker is a discrete PEG, optionally comprising from 2 to 60, from 2 to 50, or from 2 to 48 repeating ethylene oxide units. In some cases, the linker comprises a dPEG comprising about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units. In some cases, the linker is a dPEG from Quanta Biodesign, LMD.

In some embodiments, the linker is a polypeptide linker. In some instances, the polypeptide linker comprises at least 2, 3, 4, 5, 6, 7, 8, 10, 15, 20, 25, 30, 35, 40, 45, 50, 60, 70, 80, 90, 100, or more amino acid residues. In some instances, the polypeptide linker comprises at least 2, 3, 4, 5, 6, 7, 8, or more amino acid residues. In some instances, the polypeptide linker comprises at most 2, 3, 4, 5, 6, 7, 8, or less amino acid residues. In some cases, the polypeptide linker is a cleavable polypeptide linker (e.g., either enzymatically or chemically). In some cases, the polypeptide linker is a non-cleavable polypeptide linker. In some instances, the polypeptide linker comprises Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some instances, the polypeptide linker comprises a peptide such as: Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some cases, the polypeptide linker comprises L-amino acids, D-amino acids, or a mixture of both L- and D-amino acids.

In some instances, the linker comprises a homobifuctional linker. Exemplary homobifuctional linkers include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido]butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[β-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some embodiments, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(γ-maleimidobutyryloxy)succinimide ester (GMBs), N-(γ-maleimidobutyryloxy)sulfosuccinimide ester (sulfo-GMBs), succinimidyl 6-((iodoacetyl)amino)hexanoate (sIAX), succinimidyl 646-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M₂C₂H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(ρ-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(ρ-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), p-nitrophenyl diazopyruvate (pNPDP), p-nitrophenyl-2-diazo-3,3,3-trifluoropropionate (PNP-DTP), sulfhydryl-reactive and photoreactive cross-linkers such as1-(ρ-Azidosalicylamido)-4-(iodoacetamido)butane (AsIB), N-[4-(ρ-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as ρ-azidobenzoyl hydrazide (ABH), carboxylate-reactive and photoreactive cross-linkers such as 4-(ρ-azidosalicylamido)butylamine (AsBA), and arginine-reactive and photoreactive cross-linkers such as ρ-azidophenyl glyoxal (APG).

In some embodiments, the linker comprises a benzoic acid group, or its derivatives thereof. In some instances, the benzoic acid group or its derivatives thereof comprise paraaminobenzoic acid (PABA). In some instances, the benzoic acid group or its derivatives thereof comprise gamma-aminobutyric acid (GABA).

In some embodiments, the linker comprises one or more of a maleimide group, a peptide moiety, and/or a benzoic acid group, in any combination. In some embodiments, the linker comprises a combination of a maleimide group, a peptide moiety, and/or a benzoic acid group. In some instances, the maleimide group is maleimidocaproyl (mc). In some instances, the peptide group is val-cit. In some instances, the benzoic acid group is PABA. In some instances, the linker comprises a mc-val-cit group. In some cases, the linker comprises a val-cit-PABA group. In additional cases, the linker comprises a mc-val-cit-PABA group.

In some embodiments, the linker is a self-immolative linker or a self-elimination linker. In some cases, the linker is a self-immolative linker. In other cases, the linker is a self-elimination linker (e.g., a cyclization self-elimination linker). In some instances, the linker comprises a linker described in U.S. Pat. No. 9,089,614 or PCT Publication No. WO2015038426.

In some embodiments, the linker is a dendritic type linker. In some instances, the dendritic type linker comprises a branching, multifunctional linker moiety. In some instances, the dendritic type linker comprises PAMAM dendrimers.

In some embodiments, the linker is a traceless linker or a linker in which after cleavage does not leave behind a linker moiety (e.g., an atom or a linker group) to the antibody or payload. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some cases, the linker is a traceless aryl-triazene linker as described in Hejesen, et al., “A traceless aryl-triazene linker for DNA-directed chemistry,” Org Biomol Chem 11(15): 2493-2497 (2013). In some instances, the linker is a traceless linker described in Blaney, et al., “Traceless solid-phase organic synthesis,” Chem. Rev. 102: 2607-2024 (2002). In some instances, a linker is a traceless linker as described in U.S. Pat. No. 6,821,783.

### Pharmaceutical Compositions

In some embodiments, an anti-LRIG1 antibody is further formulated as a pharmaceutical composition. In some instances, the pharmaceutical composition is formulated for administration to a subject by multiple administration routes, including but not limited to, parenteral (e.g., intravenous, subcutaneous, intramuscular, intraarterial, intradermal, intraperitoneal, intravitreal, intracerebral, or intracerebroventricular), oral, intranasal, buccal, rectal, or transdermal administration routes. In some instances, the pharmaceutical composition describe herein is formulated for parenteral (e.g., intravenous, subcutaneous, intramuscular, intraarterial, intradermal, intraperitoneal, intravitreal, intracerebral, or intracerebroventricular) administration. In other instances, the pharmaceutical composition describe herein is formulated for oral administration. In still other instances, the pharmaceutical composition describe herein is formulated for intranasal administration.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some instances, the pharmaceutical compositions further include pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some instances, the pharmaceutical compositions include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some instances, the pharmaceutical compositions further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

### Therapeutic Regimens

In some embodiments, the anti-LRIG1 antibodies disclosed herein are administered for therapeutic applications. In some embodiments, the anti-LRIG1 antibody is administered once per day, twice per day, three times per day or more. The anti-LRIG1 antibody is administered daily, every day, every alternate day, five days a week, once a week, every other week, two weeks per month, three weeks per month, once a month, twice a month, three times per month, or more. The anti-LRIG1 antibody is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the anti-LRIG1 antibody is given continuously; alternatively, the dose of the anti-LRIG1 antibody being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's condition has occurred, a maintenance dose is administered if necessary. Subsequently, the dosage or the frequency of administration, or both, can be reduced, as a function of the symptoms, to a level at which the improved disease, disorder, or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages is altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Kits/Article of Manufacture

Disclosed herein, in certain embodiments, are kits and articles of manufacture for use with one or more of the compositions and methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include an anti-LRIG1 antibody as disclosed herein, host cells for producing one or more antibodies described herein, and/or vectors comprising nucleic acid molecules that encode the antibodies described herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error, e.g., within 15%, 10%, or 5%.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

The terms “polypeptide”, “peptide”, and “protein” are used interchangeably herein to refer to polymers of amino acids of any length. The polymer may be linear, cyclic, or branched, it may comprise modified amino acids, and it may be interrupted by non-amino acids. The terms also encompass amino acid polymers that have been modified, for example, via sulfation, glycosylation, lipidation, acetylation, phosphorylation, iodination, methylation, oxidation, proteolytic processing, phosphorylation, prenylation, racemization, selenoylation, transfer-RNA mediated addition of amino acids to proteins such as arginylation, ubiquitination, or any other manipulation, such as conjugation with a labeling component.

As used herein the term “amino acid” refers to either natural and/or unnatural or synthetic amino acids, including glycine and both the D or L optical isomers, and amino acid analogs and peptidomimetics.

A polypeptide or amino acid sequence “derived from” a designated protein refers to the origin of the polypeptide. Preferably, the polypeptide has an amino acid sequence that is essentially identical to that of a polypeptide encoded in the sequence, or a portion thereof wherein the portion consists of at least 10-20 amino acids, or at least 20-30 amino acids, or at least 30-50 amino acids, or which is immunologically identifiable with a polypeptide encoded in the sequence. This terminology also includes a polypeptide expressed from a designated nucleic acid sequence.

The term “humanized” as applies to a non-human (e.g. rodent or primate) antibodies are hybrid immunoglobulins, immunoglobulin chains or fragments thereof which contain minimal sequence derived from non-human immunoglobulin.

### Examples

These examples are provided for illustrative purposes only and not to limit the scope of the claims provided herein.

### Example 1. LRIG1 Binding to VISTA

This example describes assays performed to evaluate the interaction between LRIG1 and VISTA.

Binding Assays—Co-Immunoprecipitation

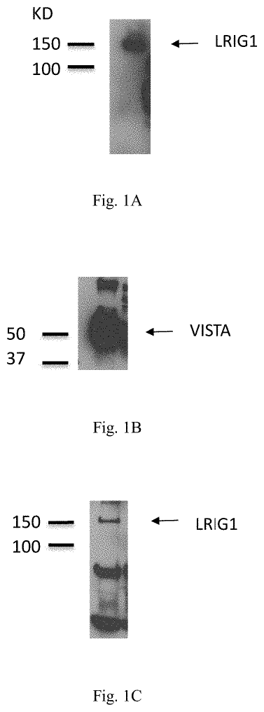

Co-immunoprecipitation experiments were performed to test whether VISTA specifically interacts with LRIG1. 293T cells were co-transfected with a plasmid encoding HA-tagged VISTA and a plasmid encoding Flag-tagged LRIG1. The transfection was performed using lipofectamine 3000 (Life Technologies) following manufacturer's protocols. The transfected cells were grown over night and then washed and lysed. The lysed cells were centrifuged and supernatant (the lysate) was collected. The lysates were prepared mixing with SDS-PAGE sample buffer and separated on SDS-PAGE. The SDS-PAGE gel was then probed with anti-Flag (FIG. 1A) and anti-HA antibodies (FIG. 1B), respectively. Both the anti-Flag and the anti-HA antibodies were purchased from Sigma. The arrows in FIG. 1A and FIG. 1B indicate the presence of LRIG1 and VISTA, respectively.

For immunoprecipitation, anti-HA antibody and protein G beads (Santa Cruz biotech) were added to the supernatant (the lysate) produced above. The beads and the lysates were incubated by rotating at 4° C. overnight to allow the HA-tagged proteins to attach. The beads were then washed 3× with lysis buffer and mixed with 1×SDS PAGE sample buffer, boiled and separated on SDS-PAGE. The SDS-PAGE gel was transferred onto a membrane, which was then probed with ant-Flag antibody (FIG. 1C). The arrow in FIG. 1C points to the presence of LRIG1, indicating that human VISTA specifically pulled down human LRIG1.

Blocking Assays—ELISA

ELISA assays were also performed to assess the interaction between VISTA and LRIG1. 96 well ELISA plates (ThermoFisher Scientific) were coated with hLRIG1 protein (R&D systems) in PBS and incubated at 4° C. for overnight. The plate was washed three times with TBST and then blocked with PBS buffer containing 2% BSA at room temperature for 1 hour. In FIG. 2, an anti hLRIG1 mAb (IMT-300) was added to well that has been coated with hLRIG1. The antibody was incubated for 10 minutes and hVISTA Fc (R&D Systems) were then added to the plates and incubated for an additional one-hour. Plates were then washed for three times and followed by incubation with anti-human-IgG-HRP (Jackson Immuno Research) for 1 hour at room temperature. The color was developed with TMB subtract (GeneTex) after three time washes with TBST and the reaction was terminated with 1N HCl. The optical density (OD) was read at 450 nm. The results were expressed as the average OD of duplicates ±SD. The results in FIG. 2 showed that monoclonal antibody IMT300 (mab4) against hLRIG1 blocked the interaction between VISTA and LRIG1 (FIG. 2).

### Example 2. LRIG1 Expression Assay

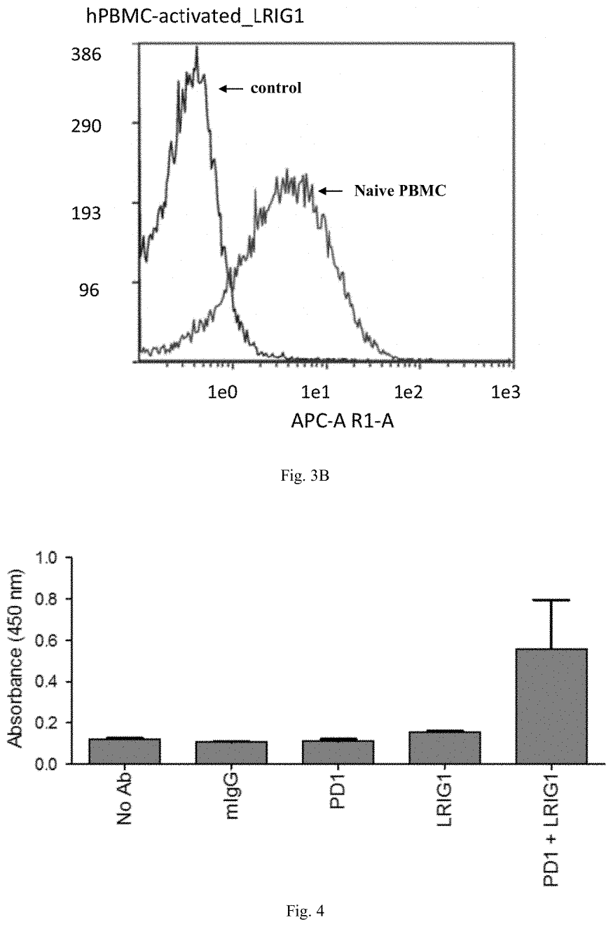

Flow cytometry was used to detect LRIG1 expression on human peripheral blood mononuclear cell (PBMC). Human PBMC was seeded on plates coated with hCD3 and hCD28 (Biolegend) for 5 days for activation. Activated or fresh PBMC was blocked with 200 μg/ml hIgG for 10 minutes on ice, followed by incubation with sheep anti-hLRIG1 polyclonal antibody (R&D Systems) or an isotype control antibody for 20 minutes on ice, followed by incubation with anti-sheep IgG APC antibody (JacksonImmuno Research) for 20 minutes on ice. After wash, stained cells were analyzed using MACSquant Analyzer 10 (Miltenyi Biosci). The results in FIGS. 3A-3B showed that LRIG1 expression was detected on activated (FIG. 3B), but not naive PBMC (light gray line) relative to isotype-control stained samples (dark gray line) (FIG. 3A).

### Example 3. LRIG1 Function-Mixed Lymphocyte Reaction (MLR)

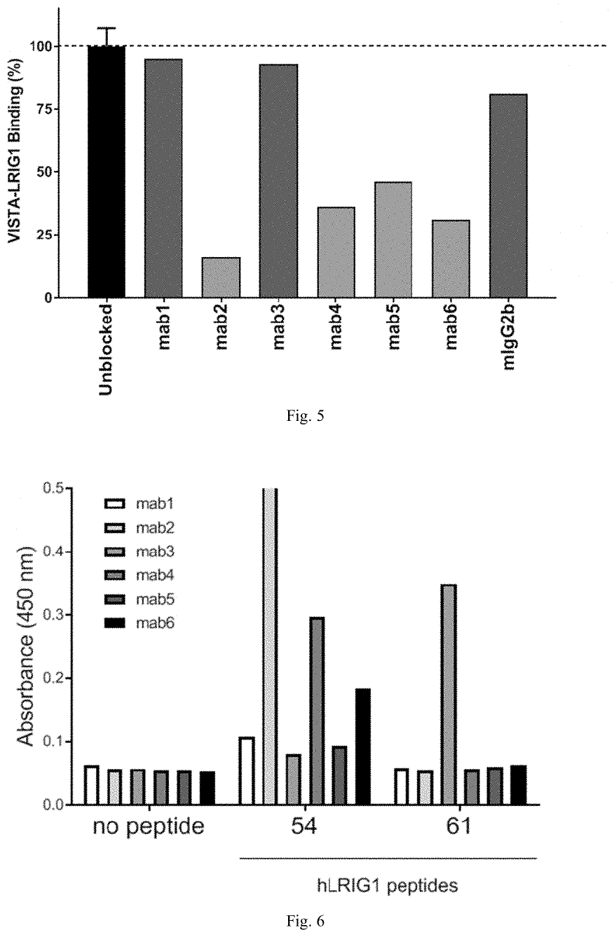

Human M2 macrophages from one donor were mixed with human CD4 T cells from another donor and were treated with 10 ug/ml control IgG, hPD1 blocking antibody EH12 (BD bioscience), hLRIG1 mAb IMT300 (mab4), or the combination of hPD1 and LRIG1 antibodies for 8 days. Secreted IFNgamma (IFNγ) was detected with an ELISA kit from eBioscience. The results in FIG. 4 showed that hLRIG1 mAb IMT300 in combination with PD1 antibody greatly enhanced the secretion of IFNγ.

### Example 4. Identification of LRIG1-Binding Antibodies with and without LRIG1-VISTA Blocking Activity

To identify LRIG1-targeted antibodies with the ability to block the interaction of LRIG1 and VISTA, purified LRIG1 and VISTA proteins were incubated in the presence of various LRIG1-targeted or control antibodies, or without antibody, and protein interaction was evaluated by ELISA. Purified human LRIG1 extracellular domain fused to a HIS tag (R&D Systems) was diluted in phosphate buffered saline (PBS) (Corning) to a concentration of 3 μg/ml and 100 ul was added to each well of a 96-well ELISA plate (Thermo Fisher, 44-2404-21). After incubating the plate at 4° C. overnight, the plate was washed three times with 300 μl of PBS with 0.05% TWEEN (VWR) (PBST) per well. The plate was then blocked for an hour with 200 μl of 2% bovine serum albumin (BSA) (Sigma) in PBST per well at room temperature with gentle rocking. Thereafter, the 2% BSA in PBST was removed and 50 ul of antibody at 3.3 ug/ml in 2% BSA in PBST was added to the wells. The plate was incubated for 10 minutes at room temperature with gentle rocking. Afterwards, 50 nM of oligomerized VISTA in 100 ul PBS buffer was added per well. VISTA oligermization was performed by Klickmer formation. Briefly, 5 nM Klickmer (Immudex) was incubated with 200 nM hVISTA-Fc-Avi-Biotin in PBS and incubated for one hour. The plate was incubated for an hour at room temperature with gentle rocking. Thereafter, the plate was washed three times with 300 μl of PBST per well, and 100 ul of avidin-HRP (1:1000) (Jackson ImmunoResearch) was then added to each well and the plate was incubated at room temperature for 30 minutes with gentle rocking. Thereafter, the plate was washed three times with 300 μl of PBST per well. 100 ul of TMB substrate (Fisher Scientific, 34029) was then added to each well. The reaction was stopped with 50 ul of 1 M HCl (VWR) per well. The plate was read using a plate reader (Molecular Devices) at absorbance of 450 nm. Percent blockade of LRIG1-VISTA interaction was calculated as the fraction of signal obtained in each experimental samples of the no antibody sample less background signal.

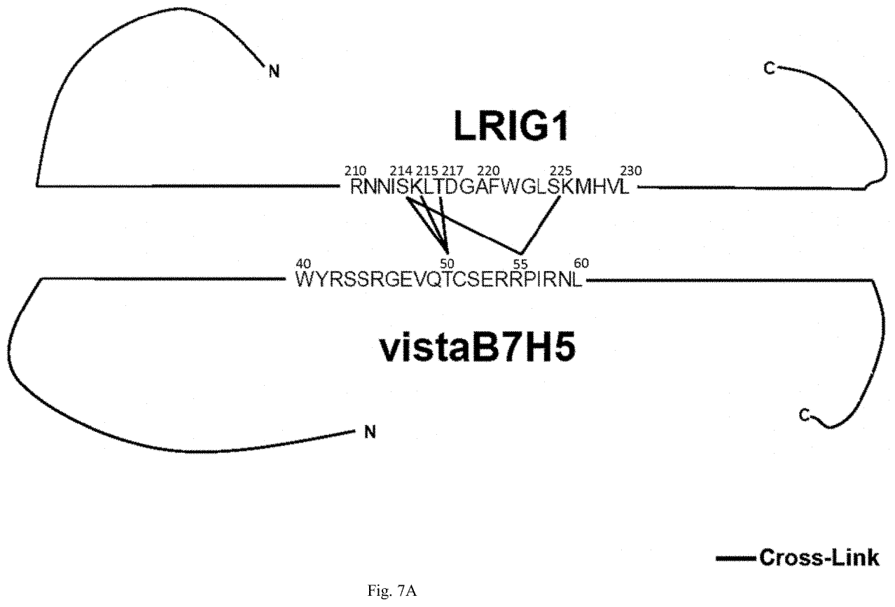

As shown in FIG. 5, LRIG1-binding antibodies exhibited differential ability to block the interaction of LRIG1 and VISTA. LRIG1-targeting mab2 strongly inhibited binding, reducing association of VISTA and LRIG1 to 21% of that observed without any antibody. mab4, mab5, and may also reduced binding of LRIG1 and VISTA to 44%, 60%, and 43%, respectively relative to an unblocked sample. In contrast, LRIG1-binding antibodies mab1 and mab3 did not significantly reduce association of LRIG1 with VISTA.

### Example 5. LRIG1-Targeted Antibodies with and without VISTA-LRIG1 Blocking Activity Bind to Distinct Epitopes of LRIG1

To identify the epitopes to which LRIG1 antibodies with and without VISTA-LRIG1 blocking activity bound, a library of 20 amino acid peptides representing portions of LRIG1 was produced, and the ability to bind LRIG1 antibodies was evaluated by ELISA. At least 2 ug/ml of hLRIG1 peptide in 50 ul of PBS or 0.1 ug/ml of full-length human LRIG1 protein (R&D Systems) in 100 ul of PBS was added to the wells of a 96-well ELISA plate (Thermo Fisher, 44-2404-21). After incubating the plate at 4° C. overnight, the plate was washed three times with 300 μl of PBST per well. The plate was then blocked for an hour with 200 μl of 2% BSA in PBST per well at room temperature with gentle rocking. Thereafter, the 2% BSA in PBST was removed and 100 ul of 0.1 ug/ml of antibody in 2% BSA in PBST was added to the wells. The plate was incubated for an hour at room temperature with gentle rocking and then washed three times with 300 μl of PBST per well. Afterwards, 100 ul of anti-mouse IgG-HRP (1:4000) (Jackson ImmunoResearch) or anti-rat IgG HRP (1:4000) (Jackson ImmunoResearch) was added to the wells. The plate was incubated for 30 minutes at room temperature with gentle rocking and then washed three times with 300 μl of PBST per well. 100 ul of TMB substrate (Fisher Scientific, 34029) was then added to each well. The reaction was stopped with 50 ul of 1 M HCl (VWR) per well. The plate was read using a plate reader (Molecular Devices) at absorbance of 450 nm.

Peptide sequences are listed in Table 4.

TABLE 4

| SEQ ID | | |
|---|---|---|
| NO. | Peptide | Sequence |
| 5 | Peptide 1 | AGPRAPCAAACTCAGDSLDC |
| 6 | Peptide 2 | CTCAGDSLDCGGRGLAALPG |
| 7 | Peptide 3 | GGRGLAALPGDLPSWTRSLN |
| 8 | Peptide 4 | DLPSWTRSLNLSYNKLSEID |
| 9 | Peptide 5 | LSYNKLSEIDPAGFEDLPNL |
| 10 | Peptide 6 | PAGFEDLPNLQEVYLNNNEL |
| 11 | Peptide 7 | QEVYLNNNELTAVPSLGAAS |
| 12 | Peptide 8 | TAVPSLGAASSHVVSLFLQH |
| 13 | Peptide 9 | SHVVSLFLQHNKIRSVEGSQ |
| 14 | Peptide 10 | NKIRSVEGSQLKAYLSLEVL |
| 15 | Peptide 11 | LKAYLSLEVLDLSLNNITEV |
| 16 | Peptide 12 | DLSLNNITEVRNTCFPHGPP |
| 17 | Peptide 13 | RNTCFPHGPPIKELNLAGNR |
| 18 | Peptide 14 | IKELNLAGNRIGTLELGAFD |
| 19 | Peptide 15 | IGTLELGAFDGLSRSLLTLR |
| 20 | Peptide 16 | GLSRSLLTLRLSKNRITQLP |
| 21 | Peptide 17 | LSKNRITQLPVRAFKLPRLT |
| 22 | Peptide 18 | VRAFKLPRLTQLDLNRNRIR |
| 23 | Peptide 19 | QLDLNRNRIRLIEGLTFQGL |
| 24 | Peptide 20 | LIEGLTFQGLNSLEVLKLQR |
| 25 | Peptide 21 | NSLEVLKLQRNNISKLTDGA |
| 26 | Peptide 22 | NNISKLTDGAFWGLSKMHVL |
| 27 | Peptide 23 | FWGLSKMHVLHLEYNSLVEV |
| 28 | Peptide 24 | HLEYNSLVEVNSGSLYGLTA |
| 29 | Peptide 25 | NSGSLYGLTALHQLHLSNNS |
| 30 | Peptide 26 | LHQLHLSNNSIARIHRKGWS |
| 31 | Peptide 27 | IARIHRKGWSFCQKLHELVL |
| 32 | Peptide 28 | FCQKLHELVLSFNNLTRLDE |
| 33 | Peptide 29 | SFNNLTRLDEESLAELSSLS |
| 34 | Peptide 30 | ESLAELSSLSVLRLSHNSIS |
| 35 | Peptide 31 | VLRLSHNSISHIAEGAFKGL |
| 36 | Peptide 32 | HIAEGAFKGLRSLRVLDLDH |
| 37 | Peptide 33 | RSLRVLDLDHNEISGTIEDT |
| 38 | Peptide 34 | NEISGTIEDTSGAFSGLDSL |
| 39 | Peptide 35 | SGAFSGLDSLSKLTLFGNKI |
| 40 | Peptide 36 | SKLTLFGNKIKSVAKRAFSG |
| 41 | Peptide 37 | KSVAKRAFSGLEGLEHLNLG |
| 42 | Peptide 38 | LEGLEHLNLGGNAIRSVQFD |
| 43 | Peptide 39 | GNAIRSVQFDAFVKMKNLKE |
| 44 | Peptide 40 | AFVKMKNLKELHISSDSFLC |
| 45 | Peptide 41 | LHISSDSFLCDCQLKWLPPW |
| 46 | Peptide 42 | DCQLKWLPPWLIGRMLQAFV |
| 47 | Peptide 43 | LIGRMLQAFVTATCAHPESL |
| 48 | Peptide 44 | TATCAHPESLKGQSIFSVPP |
| 49 | Peptide 45 | KGQSIFSVPPESFVCDDFLK |
| 50 | Peptide 46 | ESFVCDDFLKPQIITQPETT |
| 51 | Peptide 47 | PQIITQPETTMAMVGKDIRF |
| 52 | Peptide 48 | MAMVGKDIRFTCSAASSSSS |
| 53 | Peptide 49 | TCSAASSSSSPMTFAWKKDN |
| 54 | Peptide 50 | PMTFAWKKDNEVLTNADMEN |
| 55 | Peptide 51 | EVLTNADMENFVHVHAQDGE |
| 56 | Peptide 52 | FVHVHAQDGEVMEYTTILHL |
| 57 | Peptide 53 | VMEYTTILHLRQVTFGHEGR |
| 58 | Peptide 54 | RQVTFGHEGRYQCVITNHFG |
| 59 | Peptide 55 | YQCVITNHFGSTYSHKARLT |
| 60 | Peptide 56 | STYSHKARLTVNVLPSFTKT |
| 61 | Peptide 57 | VNVLPSFTKTPHDITIRTTT |
| 62 | Peptide 58 | PHDITIRTTTMARLECAATG |
| 63 | Peptide 59 | MARLECAATGHPNPQIAWQK |
| 64 | Peptide 60 | HPNPQIAWQKDGGTDFPAAR |
| 65 | Peptide 61 | DGGTDFPAARERRMHVMPDD |
| 66 | Peptide 62 | ERRMHVMPDDDVFFITDVKI |
| 67 | Peptide 63 | DVFFITDVKIDDAGVYSCTA |
| 68 | Peptide 64 | DDAGVYSCTAQNSAGSISAN |
| 69 | Peptide 65 | QNSAGSISANATLTVLETPS |
| 70 | Peptide 66 | ATLTVLETPSLVVPLEDRVV |
| 71 | Peptide 67 | LVVPLEDRVVSVGETVALQC |
| 72 | Peptide 68 | SVGETVALQCKATGNPPPRI |
| 73 | Peptide 69 | KATGNPPPRITWFKGDRPLS |
| 74 | Peptide 70 | TWFKGDRPLSLTERHHLTPD |
| 75 | Peptide 71 | LTERHHLTPDNQLLVVQNVV |
| 76 | Peptide 72 | NQLLVVQNVVAEDAGRYTCE |
| 77 | Peptide 73 | AEDAGRYTCEMSNTLGTERA |
| 78 | Peptide 74 | MSNTLGTERAHSQLSVLPAA |
| 79 | Peptide 75 | HSQLSVLPAAGCRKDGTTVG |
| 80 | Peptide 76 | GCRKDGTTVG |

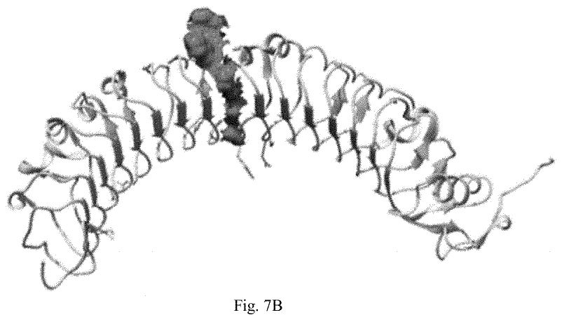

As shown in FIG. 6, LRIG1-targeted antibodies with VISTA-LRIG1 blocking activity mab2, mab4, and mab6, bound to peptide 54, corresponding to amino acids 565-584 of SEQ ID NO: 2. Separately, mab3, which showed no VISTA-LRIG1 blocking activity bound to peptide 61, corresponding to amino acids 635-654 of SEQ ID NO: 2. mab1, which also lacked VISTA-LRIG1 blocking activity did not bind any LRIG1 peptide, suggesting poor affinity or a non-linear epitope for this antibody. Similarly, mab5, which most weakly blocked LRIG1 and VISTA binding failed to bind any LRIG1 peptides, suggesting poor affinity or a non-linear epitope. Peptide 54 illustrates an epitope of LRIG1 for determining antibodies with VISTA-LRIG1 blocking activity.

### Example 6. LRIG1-VISTA Antibodies with Blocking Activity Compete for Binding to LRIG1

To determine whether LRIG1 binding antibodies with LRIG1-VISTA blocking activity bind to the same or overlapping regions of the LRIG1 molecule, antibody binning assays were performed to assess the ability of antibodies to bind simultaneously bind LRIG1 Amine-reactive probes were loaded onto a Gator biosensor (Probe Life, Palo Alto, Calif.), equilibrated in dH20 for 60 seconds, dipped into 100 ul EDC 0.2M/NHS 0.05M activation buffer for 30 seconds, then dipped into a solution of 20 ug/ul human LRIG1-His in 10 mM NaOAc buffer, pH 5 until binding was saturated and quenched in 1M ethanolamine pH 8.5 for 300 seconds. Following LRIG1-His loading, tips were dipped in 20 ug/mL saturating antibody, then successively dipped into 5 ug/mL competing antibody.

As depicted in Tables 5-6, saturation of hLRIG1-His tips with any individual antibody prevented binding with the same antibody in a competition study. Competition between pairs of distinct antibodies revealed one class of competitive bins. mab2, mab4, mab5, and mab6, exhibited mutual competitive binding for hLRIG1-His, but did not compete with mab1 or mab3, thus defining bin A. The competition observed of mab5 with mab2, mab4, and mab6 was unanticipated in view of its failure to significantly bind peptide 54, whereas mab2, mab4, and mab6 did bind peptide 54. In contrast, mab1 and mab3 failed to bin with each other or any other antibody. Significantly, antibodies binding in bin A correlated with those the ability to block the association of LRIG1 and VISTA as depicted in FIG. 5, whereas the unbinned antibodies mab1 and mab3 failed to block this interaction. Accordingly, the ability of antibodies to compete for binding with mab2, mab4, mab5, and mab6, defining bin A, is predictive of the ability of the same antibodies to disrupt interaction of VISTA and LRIG1.

TABLE 5

| | | Competing Ab | | | | | |
|---|---|---|---|---|---|---|---|
| | | Ab1 | Ab2 | Ab3 | Ab4 | Ab5 | Ab6 |
| | | (mab1) | (mab2) | (mab3) | (mab4) | (mab5) | (mab6) |
| Satu- | Ab1 | NB | NB | NB | NB | NB | NB |
| rating | (mab1) | | | | | | |
| Ab | Ab2 | NB | 0 | NB | 0 | 0 | 0 |
| | (mab2) | | | | | | |
| | Ab3 | NB | NB | NB | NB | NB | NB |
| | (mab3) | | | | | | |
| | Ab4 | NB | 0 | NB | 0 | 0 | 0 |
| | (mab4) | | | | | | |
| | Ab5 | NB | 0 | NB | 0 | 0 | 0 |
| | (mab5) | | | | | | |
| | Ab6 | NB | 0 | NB | 0 | 0 | 0 |
| | (mab6) | | | | | | |

TABLE 6

| Bin | Antibody |
|---|---|
| Unbinned | mab1, mab3 |
| Bin A | mab2, mab4, mab5, mab6 |

### Example 7. Identification of VISTA-LRIG1 Binding Surface

To identify the residues mediating the interaction between LRIG1 and VISTA, a crosslinked mass spectroscopy approach was used. 5 ul of purified 3.2 uM LRIG1 and 5 ul of purified 0.6 uM VISTA were mixed and were submitted to cross-linking with a K200 MALDI MS analysis kit (CovalX). 9 μl of the mixture was mixed with 1 μl of K200 Stabilizer reagent (2 mg/ml) and incubated at room temperature. After the incubation time (180 minutes) the samples were prepared for MALDI analysis as for Control experiments. The samples were analyzed by High-Mass MALDI analysis immediately after crystallization. For the analysis, the following parameters have been applied: Mass Spectrometer: Linear and Positive mode, Ion Source 1: 20 kV, Ion Source 2: 17 kV, Lens: 12 kV, Pulse Ion Extraction: 400 ns HM4:Gain Voltage: 3.14 kV, Acceleration Voltage: 20 kV. Crosslinked LRIG1-VISTA products were identified with MH+=207.154 kDa. Samples were digested with Trypsin, chymotrypsin, ASPN-N, Elastase, or Thermolysin and crosslinked peptides with both LRIG1 and VISTA amino acid sequences were determined.

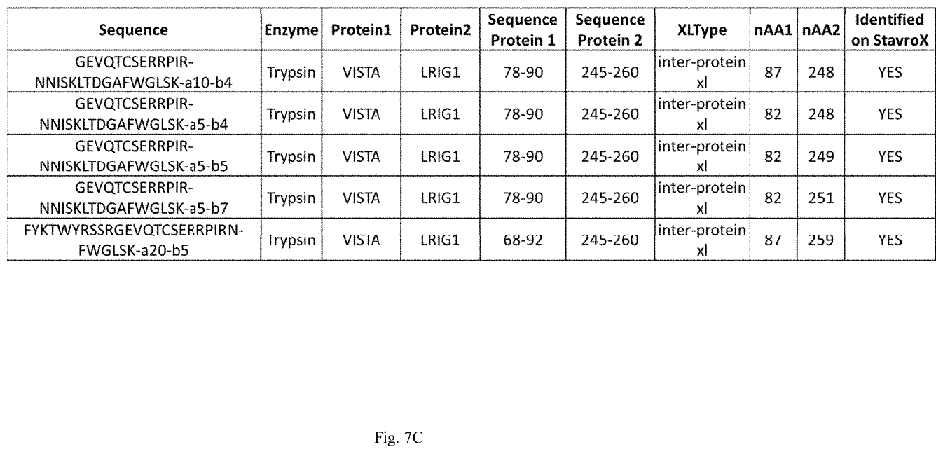

As depicted in FIGS. 7A-7C, residues in the vicinity of LRIG1 amino acids 245-260 were found to be crosslinked to residues in the vicinity of VISTA amino acids 68-92. These amino acids are located on exposed regions of each molecule, suggesting that these regions are involved in the protein-protein interaction of LRIG1 and VISTA. It is notable that the LRIG1 binding interface at amino acids 246-260 determined by MALDI-MS is distinct from the epitope bound by the LRIG1-VISTA blocking antibodies mab2, mab4, and mab6. The binding of these antibodies may induce a conformational shift that causes a structural rearrangement, thereby impacting binding. Identification of a distinct binding interface mediated by of LRIG1 amino acids 245-260 suggests that antibodies which bind a region other than defined by peptide 52 could also disrupt the interaction of LRIG1 and VISTA.

### Example 8. LRIG1-VISTA Blockade Reduces Tumor Growth in a Humanized Mouse Tumor Model

To evaluate the utility of LRIG1-VISTA blockade in the setting of cancer, mice engrafted with a human immune system and bearing human SCLC tumors were employed. All animal studies were conducted in compliance with the U.S. Department of Agriculture's Animal Welfare Act (9 CFR Parts 1, 2 and 3) as applicable and were covered by an IACUC approved animal protocol. Briefly, NOD.Cg-Prkdc^{scid} Il2rg^{tm1Sug} Tg(SV40/HTLV-IL3,CSF2)10-7Jic/JicTac mice, also known as NOG-EXL mice (Taconic), were engrafted with human CD34+ hematopoietic stem cells, and 50,000 human small cell lung cancer (SCLC) patient derived xenograft (PDX) model LU5173 tumor cells mixed with an equal volume of Cultrex ECM (Trevigen) in 100 ul total volume were subcutaneously injected into the rear flank with a chilled 1 ml Luer-lok syringe fitted with a 26G 7/8 (0.5 mm×22 mm) needle Animals were monitored weekly for palpable tumors, or any changes in appearance or behavior and daily monitoring was conducted for mice showing any signs of morbidity or mortality. Tumor volume was calculated using the following equation: (longest diameter*shortest diameter²)/2. When average tumor volume reached 60-100 mm³, 12 mice were randomly assigned to the respective treatment groups receiving either A) HuIgG4 control antibody, BIW×3 weeks at 10 mg/kg; B) Anti-PD1 OPDIVO antibody, BIW×3 weeks at 10 mg/kg or; C) anti-LRIG1 antibody IMT300 (mab4) BIW×3 weeks at 10 mg/kg.

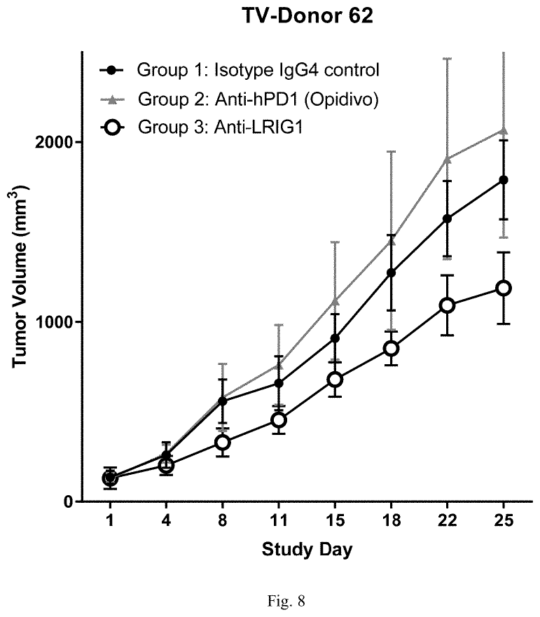

As shown in FIG. 8 and Table 7, tumor growth in animals treated with huIgG4 control antibody grew to a mean tumor volume of 1760 mm³ after 25 days of treatment, whereas tumors in animals treated with Opdivo grew to a mean volume of 2068 mm³, reflecting tumor growth inhibition (TGI) of −16%. In contrast, tumors in animals treated with IMT300 grew to only 1188 mm³ in this same period, reflecting a TGI of 34%. Further, IMT300-treated animals exhibited a significantly reduced terminal growth kinetic of 32 mm³/day relative to control treated animals at 72 mm³/day. Collectively, these data indicate that blockade of LRIG1-VISTA with LRIG1-binding antibody IMT300 can inhibit human tumor growth with greater efficacy than the PD1-PDL1 blocking antibody OPDIVO.

TABLE 7

| Treatment | TGI |
|---|---|
| IgG4 control | — |
| anti-PD1 Opdivo | −16% |
| anti-LRIG1 IMT300 | 34% |

### Example 9. Antibodies Used

Table 8 below lists antibody information for studies described herein.

| Ab | Manufacturer | Catalog # |
|---|---|---|
| mab1 | EMD Millipore | MAB S1816 |
| mab2 | R & D Systems | MAB7498 |
| mab3 | Santa Cruz | sc-514577 |
| mab4 | Immutics | PP14384 |
| (IMT300) | | |
| mab5 | Immutics | PP14385 |
| mab6 | Immutics | PP14387 |

### Example 10. Sequences

Table 9 below illustrates sequences described above.

| SEQ | | |
|---|---|---|
| ID | | |
| NO. | Name | Sequence |
| 1 | LRIG1 | gcgctccagacaagATGgcgcggccggtccggggagggctcggggccccgcgccgctcgccttgc |
| | nucleic | cttctccttctctggctgcttttgcttcggctggagccggtgaccgccgcggccggcccgcgggcgccctgc |
| | acid | gcggccgcctgcacttgcgctggggactcgctggactgcggtgggcgcgggctggctgcgttgcccggg |
| | (cDNA) | gacctgccctcctggacgcggagcctaaacctgagttacaacaaactctctgagattgaccctgctggttttg |
| | (homo | aggacttgccgaacctacaggaagtgtacctcaataataatgagttgacagcggtaccatccctgggcgct |
| | sapiens) | gcttcatcacatgtcgtctctctctttctgcagcacaacaagattcgcagcgtggaggggagccagctgaag |
| | | gcctacctttccttagaagtgttagatctgagtttgaacaacatcacggaagtgcggaacacctgctttccaca |
| | | cggaccgcctataaaggagctcaacctggcaggcaatcggattggcaccctggagttgggagcatttgatg |
| | | gtctgtcacggtcgctgctaactcttcgcctgagcaaaaacaggatcacccagcttcctgtaagagcattcaa |
| | | gctacccaggctgacacaactggacctcaatcggaacaggattcggctgatagagggcctcaccttccag |
| | | gggctcaacagcttggaggtgctgaagcttcagcgaaacaacatcagcaaactgacagatggggccttct |
| | | ggggactgtccaagatgcatgtgctgcacctggagtacaacagcctggtagaagtgaacagcggctcgct |
| | | ctacggcctcacggccctgcatcagctccacctcagcaacaattccatcgctcgcattcaccgcaagggct |
| | | ggagcttctgccagaagctgcatgagttggtcctgtccttcaacaacctgacacggctggacgaggagagc |
| | | ctggccgagctgagcagcctgagtgtcctgcgtctcagccacaattccatcagccacattgcggagggtgc |
| | | cttcaagggactcaggagcctgcgagtcttggatctggaccataacgagatttcgggcacaatagaggaca |
| | | cgagcggcgccttctcagggctcgacagcctcagcaagctgactctgtttggaaacaagatcaagtctgtg |
| | | gctaagagagcattctcggggctggaaggcctggagcacctgaaccttggagggaatgcgatcagatctg |
| | | tccagtttgatgcctttgtgaagatgaagaatcttaaagagctccatatcagcagcgacagcttcctgtgtgac |
| | | tgccagctgaagtggctgcccccgtggctaattggcaggatgctgcaggcctttgtgacagccacctgtgc |
| | | ccacccagaatcactgaagggtcagagcattttctctgtgccaccagagagtttcgtgtgcgatgacttcctg |
| | | aagccacagatcatcacccagccagaaaccaccatggctatggtgggcaaggacatccggtttacatgctc |
| | | agcagccagcagcagcagctcccccatgacctttgcctggaagaaagacaatgaagtcctgaccaatgca |
| | | gacatggagaactttgtccacgtccacgcgcaggacggggaagtgatggagtacaccaccatcctgcacc |
| | | tccgtcaggtcactttcgggcacgagggccgctaccaatgtgtcatcaccaaccactttggctccacctattc |
| | | acataaggccaggctcaccgtgaatgtgttgccatcattcaccaaaacgccccacgacataaccatccgga |
| | | ccaccaccatggcccgcctcgaatgtgctgccacaggtcacccaaaccctcagattgcctggcagaagga |
| | | tggaggcacggatttccccgctgcccgtgagcgacgcatgcatgtcatgccggatgacgacgtgtttttcat |
| | | cactgatgtgaaaatagatgacgcaggggtttacagctgtactgctcagaactcagccggttctatttcagct |
| | | aatgccaccctgactgtcctagagaccccatccttggtggtccccttggaagaccgtgtggtatctgtggga |
| | | gaaacagtggccctccaatgcaaagccacggggaaccctccgccccgcatcacctggttcaagggggac |
| | | cgcccgctgagcctcactgagcggcaccacttgacccctgacaaccagctcctggtggttcagaacgtggt |
| | | ggcagaggatgcgggccgatatacctgtgagatgtccaacaccctgggcacggagcgagctcacagcca |
| | | gctgagcgtcctgcccgcagcaggctgcaggaaggatgggaccacggtaggcatcttcaccattgctgtc |
| | | gtgagcagcatcgtcctgacgtcactggtctgggtgtgcatcatctaccagaccaggaagaagagtgaaga |
| | | gtacagtgtcaccaacacagatgaaaccgtcgtgccaccagatgttccaagctacctctcactcaggggac |
| | | cctttctgaccgacaagaaaccgtggtcaggaccgagggtggccctcaggccaatgggcacattgagagc |
| | | aatggtgtgtgtccaagagatgcaagccactttccagagcccgacactcacagcgttgcctgcaggcagcc |
| | | aaagctctgtgctgggtctgcgtatcacaaagagccgtggaaagcgatggagaaagctgaagggacacct |
| | | gggccacataagatggaacacggtggccgggtcgtatgcagtgactgcaacaccgaagtggactgttact |
| | | ccaggggacaagccttccacccccagcctgtgtccagagacagcgcacagccaagtgcgccaaatggcc |
| | | cggagccgggtgggagtgaccaagagcattctccacatcaccagtgcagcaggactgccgctgggtcct |
| | | gccccgagtgccaagggtcgctctaccccagtaaccacgatagaatgctgacggctgtgaagaaaaagcc |
| | | aatggcatctctagatgggaaaggggattcacctggactttagcaaggttgtatcacccggactccacaga |
| | | gctacagcctgcatcttcattaacttcaggcagtccagagcgcgcggaagcccagtacttgcttgtttccaat |
| | | ggccacctccccaaagcatgtgacgccagtcccgagtccacgccactgacaggacagctccccgggaaa |
| | | cagagggtgccactgctgttggcaccaaaaagcTAGgttttgtctacctcagttcttgtcataccaatctct |
| | | acgggaaagagaggtaggagaggctgcgaggaagcttgggttcaagcgtcactcatctgtacatagttgta |
| | | actcccatgtggagtatcagtcgctcacaggacttggatctgaagcacagtaaacgcaagaggggatttgtg |
| | | tacaaaaggcaaaaaaagtatttgatatcattgtacataagagttttcagagatttcatatatatcttttacagag |
| | | gctattttaatctttagtgcatggttaacagaaaaaaattatacaattttgacaatattatttttcgtatcaggttgct |
| | | gtttaattttggagggggtggggaaatagttctggtgccttaacgcatggctggaatttatagaggctacaac |
| | | cacatttgttcacaggagtttttggtgcggggtgggaaggatggaaggccttggatttatattgcacttcatag |
| | | acccctaggctgctgtgcggtgggactccacatgcgccggaaggagcttcaggtgagcactgctcatgtgt |
| | | ggatgcccctgcaacaggcttccctgtctgtagagccaggggtgcaagtgccatccacacttgcagtgaat |
| | | ggcttttccttttaggtttaagtcctgtctgtctgtaaggcgtagaatctgtccgtctgtaaggcgtagaatgagg |
| | | gttgttaatccatcacaagcaaaaggtcagaacagttaaacactgcctttcctcctcctcttattttatgataaaa |
| | | gcaaatgtggccttctcagtatcattcgattgctatttgagacttttaaattaaggtaaaggctgctggtgttggt |
| | | acctgtggatttttctatactgatgttttcgttttgccaatataatgagtattacattggccttgggggacagaaag |
| | | gaggaagttctgacttttcagggctaccttatttctactaaggacccagagcaggcctgtccatgccattcctt |
| | | cgcacagatgaaactgagctgggactggaaaggacagcccttgacctgggttctgggtataatttgcactttt |
| | | gagactggtagctaaccatcttatgagtgccaatgtgtcatttagtaaaacttaaatagaaacaaggtccttca |
| | | aatgacctttggccaaaagctgaagggagttactgagaaaatagttaacaattactgtcaggtgtcatcactg |
| | | ttcaaaaggtaagcacatttagaattttgacttgacagttaactgactaatcttacttccacaaaatatgtgaattt |
| | | gctgcttctgagaggcaatgtgaaagagggagtattacttttatgtacaaagttatttatttatagaaattttggta |
| | | cagtgtacattgaaaaccatgtaaaatattgaagtgtctaacaaatggcattgaagtgtctttaataaaggttca |
| | | tttataaatgtcaaaaaaaaaaaaaaaaaaaaaaaaaaaaaaaaaaaaaaaaaaaaaaaaaaa |
| 2 | LRIG1 | MARPVRGGLGAPRRSPCLLLLWLLLLRLEPVTAAAGPRAPCAAACT |
| | protein | CAGDSLDCGGRGLAALPGDLPSWTRSLNLSYNKLSEIDPAGFEDLP |
| | sequence | NLQEVYLNNNELTAVPSLGAASSHVVSLFLQHNKIRSVEGSQLKAY |
| | (homo | LSLEVLDLSLNNITEVRNTCFPHGPPIKELNLAGNRIGTLELGAFDGL |
| | sapiens) | SRSLLTLRLSKNRITQLPVRAFKLPRLTQLDLNRNRIRLIEGLTFQGL |
| | | NSLEVLKLQRNNISKLTDGAFWGLSKMHVLHLEYNSLVEVNSGSL |
| | | YGLTALHQLHLSNNSIARIHRKGWSFCQKLHELVLSFNNLTRLDEES |
| | | LAELSSLSVLRLSHNSISHIAEGAFKGLRSLRVLDLDHNEISGTIEDTS |
| | | GAFSGLDSLSKLTLFGNKIKSVAKRAFSGLEGLEHLNLGGNAIRSVQ |
| | | FDAFVKMKNLKELHISSDSFLCDCQLKWLPPWLIGRMLQAFVTATC |
| | | AHPESLKGQSIFSVPPESFVCDDFLKPQIITQPETTMAMVGKDIRFTC |
| | | SAASSSSSPMTFAWKKDNEVLTNADMENFVHVHAQDGEVMEYTTI |
| | | LHLRQVTFGHEGRYQCVITNHFGSTYSHKARLTVNVLPSFTKTPHDI |
| | | TIRTTTMARLECAATGHPNPQIAWQKDGGTDFPAARERRMHVMPD |
| | | DDVFFITDVKIDDAGVYSCTAQNSAGSISANATLTVLETPSLVVPLE |
| | | DRVVSVGETVALQCKATGNPPPRITWFKGDRPLSLTERHHLTPDNQ |
| | | LLVVQNVVAEDAGRYTCEMSNTLGTERAHSQLSVLPAAGCRKDGT |
| | | TVGIFTIAVVSSIVLTSLVWVCIIYQTRKKSEEYSVTNTDETVVPPDV |
| | | PSYLSSQGTLSDRQETVVRTEGGPQANGHIESNGVCPRDASHFPEPD |
| | | THSVACRQPKLCAGSAYHKEPWKAMEKAEGTPGPHKMEHGGRVV |
| | | CSDCNTEVDCYSRGQAFHPQPVSRDSAQPSAPNGPEPGGSDQEHSP |
| | | HHQCSRTAAGSCPECQGSLYPSNHDRMLTAVKKKPMASLDGKGDS |
| | | SWTLARLYHPDSTELQPASSLTSGSPERAEAQYLLVSNGHLPKACD |
| | | ASPESTPLTGQLPGKQRVPLLLAPKS |
| 3 | VISTA | gggggcgggtgcctggagcacggcgctggggccgcccgcagcgctcactcgctcgcactcagtcgcgg |
| | nucleic | gaggcttccccgcgccggccgcgtcccgcccgctccccggcaccagaagttcctctgcgcgtccgacgg |
| | acid | cgacATGggcgtccccacggccctggaggccggcagctggcgctggggatccctgctcttcgctctct |
| | (cDNA) | tcctggctgcgtccctaggtccggtggcagccttcaaggtcgccacgccgtattccctgtatgtctgtcccga |
| | (homo | ggggcagaacgtcaccctcacctgcaggctcttgggccctgtggacaaagggcacgatgtgaccttctac |
| | sapiens) | aagacgtggtaccgcagctcgaggggcgaggtgcagacctgctcagagcgccggcccatccgcaacct |
| | | cacgttccaggaccttcacctgcaccatggaggccaccaggctgccaacaccagccacgacctggctcag |
| | | cgccacgggctggagtcggcctccgaccaccatggcaacttctccatcaccatgcgcaacctgaccctgct |
| | | ggatagcggcctctactgctgcctggtggtggagatcaggcaccaccactcggagcacagggtccatggt |
| | | gccatggagctgcaggtgcagacaggcaaagatgcaccatccaactgtgtggtgtacccatcctcctccca |
| | | ggatagtgaaaacatcacggctgcagccctggctacgggtgcctgcatcgtaggaatcctctgcctccccc |
| | | tcatcctgctcctggtctacaagcaaaggcaggcagcctccaaccgccgtgcccaggagctggtgcggat |
| | | ggacagcaacattcaagggattgaaaaccccggctttgaagcctcaccacctgcccaggggatacccgag |
| | | gccaaagtcaggcaccccctgtcctatgtggcccagcggcagccttctgagtctgggcggcatctgctttcg |
| | | gagcccagcacccccctgtctcctccaggccccggagacgtcttcttcccatccctggaccctgtccctgac |
| | | tctccaaactttgaggtcatcTAGcccagctgggggacagtgggctgagtggctgggtctggggcagg |
| | | tgcatttgagccagggctggctctgtgagtggcctccttggcctcggccctggttccctccctcctgctctgg |
| | | gctcagatactgtgacatcccagaagcccagcccctcaacccctctggatgctacatggggatgctggacg |
| | | gctcagcccctgttccaaggattttggggtgctgagattctcccctagagacctgaaattcaccagctacaga |
| | | tgccaaatgacttacatcttaagaagtctcagaacgtccagcccttcagcagctctcgttctgagacatgagc |
| | | cttgggatgtggcagcatcagtgggacaagatggacactgggccaccctcccaggcaccagacacaggg |
| | | cacggtggagagacttctcccccgtggccgccttggctcccccgattgcccgaggctgctcttctgtcaga |
| | | cttcctctttgtaccacagtggctctggggccaggcctgcctgcccactggccatcgccaccttccccagct |
| | | gcctcctaccagcagtttctctgaagatctgtcaacaggttaagtcaatctggggcttccactgcctgcattcc |
| | | agtccccagagcttggtggtcccgaaacgggaagtacatattggggcatggtggcctccgtgagcaaatg |
| | | gtgtcttgggcaatctgaggccaggacagatgttgccccacccactggagatggtgctgagggaggtggg |
| | | tggggccttctgggaaggtgagtggagaggggcacctgccccccgccctccccatcccctactcccactg |
| | | ctcagcgcgggccattgcaagggtgccacacaatgtcttgtccaccctgggacacttctgagtatgaagcg |
| | | ggatgctattaaaaactacatggggaaacaggtgcaaaccctggagatggattgtaagagccagtttaaatc |
| | | tgcactctgctgctcctcccccacccccaccttccactccatacaatctgggcctggtggagtcttcgcttcag |
| | | agccattcggccaggtgcgggtgatgacccatctcctgcttgtgggcatgccctggctttgatttttatacacat |
| | | aggcaaggtgagtcctctgtggaattgtgattgaaggattttaaagcaggggaggagagtagggggcatct |
| | | ctgtacactctgggggtaaaacagggaaggcagtgcctgagcatggggacaggtgaggtggggctggg |
| | | cagaccccctgtagcgtttagcaggatgggggccccaggtactgtggagagcatagtccagcctgggcat |
| | | ttgtctcctagcagcctacactggctctgctgagctgggcctgggtgctgaaagccaggatttggggctagg |
| | | cgggaagatgttcgcccaattgcttggggggttggggggatggaaaaggggagcacctctaggctgcctg |
| | | gcagcagtgagccctgggcctgtggctacagccagggaaccccacctggacacatggccctgcttctaag |
| | | ccccccagttaggcccaaaggaatggtccactgagggcctcctgctctgcctgggctgggccaggggcttt |
| | | gaggagagggtaaacataggcccggagatggggctgacacctcgagtggccagaatatgcccaaaccc |
| | | cggcttctcccttgtccctaggcagaggggggtcccttcttttgaccctctggtcaccacaatgcttgatgcca |
| | | gctgccataggaagagggtgctggctggccatggtggcacacacctgtcctcccagcactttgcagggct |
| | | gaggtggaaggaccgcttaagcccaggtgttcaaggctgctgtgagctgtgttcgagccactacactccag |
| | | cctggggacggagcaaaactttgcctcaaaacaaattttaaaaagaaagaaagaaggaaagagggtatgtt |
| | | tttcacaattcatgggggcctgcatggcaggagtggggacaggacacctgctgttcctggagtcgaaggac |
| | | aagcccacagcccagattccggttctcccaactcaggaagagcatgccctgccctctggggaggctggcc |
| | | tggccccagccctcagctgctgaccttgaggcagagacaacttctaagaatttggctgccagaccccaggc |
| | | ctggctgctgctgtgtggagagggaggcggcccgcagcagaacagccaccgcacttcctcctcagcttcc |
| | | tctggtgcggccctgccctctcttctctggacccttttacaactgaacgcatctgggcttcgtggtttcctgttttc |
| | | agcgaaatttactctgagctcccagttccatcttcatccatggccacaggccctgcctacaacgcactaggg |
| | | acgtccctccctgctgctgctggggaggggcaggctgctggagccgccctctgagttgcccgggatggta |
| | | gtgcctctgatgccagccctggtggctgtgggctggggtgcatgggagagctgggtgcgagaacatggc |
| | | gcctccagggggcgggaggagcactaggggctggggcaggaggctcctggagcgctggattcgtggc |
| | | acagtctgaggccctgagagggaaatccatgcttttaagaactaattcattgttaggagatcaatcaggaatta |
| | | ggggccatcttacctatctcctgacattcacagtttaatagagacttcctgcctttattccctcccagggagagg |
| | | ctgaaggaatggaattgaaagcaccatttggagggttttgctgacacagcggggactgctcagcactcccta |
| | | aaaacacaccatggaggccactggtgactgctggtgggcaggctggccctgcctgggggagtccgtggc |
| | | gatgggcgctggggtggaggtgcaggagccccaggacctgcttttcaaaagacttctgcctgaccagagc |
| | | tcccactacatgcagtggcccagggcagaggggctgatacatggcctttttcagggggtgctcctcgcggg |
| | | gtggacttgggagtgtgcagtgggacagggggctgcaggggtcctgccaccaccgagcaccaacttggc |
| | | ccctggggtcctgcctcatgaatgaggccttccccagggctggcctgactgtgctgggggctgggttaacg |
| | | ttttctcagggaaccacaatgcacgaaagaggaactggggttgctaaccaggatgctgggaacaaaggcc |
| | | tcttgaagcccagccacagcccagctgagcatgaggcccagcccatagacggcacaggccacctggccc |
| | | attccctgggcattccctgctttgcattgctgcttctcttcaccccatggaggctatgtcaccctaactatcctgg |
| | | aatgtgttgagagggattctgaatgatcaatatagcttggtgagacagtgccgagatagatagccatgtctgc |
| | | cttgggcacgggagagggaagtggcagcatgcatgctgtttcttggccttttctgttagaatacttggtgctttc |
| | | caacacactttcacatgtgttgtaacttgtttgatccacccccttccctgaaaatcctgggaggttttattgctgc |
| | | catttaacacagagggcaatagaggttctgaaaggtctgtgtcttgtcaaaacaagtaaacggtggaactac |
| | | gactaaa |
| 4 | VISTA | MGVPTALEAGSWRWGSLLFALFLAASLGPVAAFKVATPYSLYVCP |
| | protein | EGQNVTLTCRLLGPVDKGHDVTFYKTWYRSSRGEVQTCSERRPIRN |
| | sequence | LTFQDLHLHHGGHQAANTSHDLAQRHGLESASDHHGNFSITMRNL |
| | (homo | TLLDSGLYCCLVVEIRHHHSEHRVHGAMELQVQTGKDAPSNCVVY |
| | sapiens) | PSSSQDSENITAAALATGACIVGILCLPLILLLVYKQRQAASNRRAQE |
| | | LVRMDSNIQGIENPGFEASPPAQGIPEAKVRHPLSYVAQRQPSESGR |
| | | HLLSEPSTPLSPPGPGDVFFPSLDPVPDSPNFEVI |

* start and stop codons are in upper case and underlined

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of disrupting an interaction between VISTA and LRIG1, comprising:
contacting a plurality of cells comprising a LRIG1-expressing cell, a VISTA-expressing cell, or a combination thereof with an antibody that specifically binds to LRIG1.

2. The method of claim 1, wherein the LRIG1-VISTA interaction is reduced to less than 80%, less than 78%, less than 70%, less than 72%, less than 66%, less than 60%, less than 56%, less than 54%, less than 52%, less than 50%, less than 44%, less than 43%, less than 40%, less than 30%, less than 29%, less than 27%, less than 21%, less than 20%, less than 19%, less than 17%, less than 10%, less than 5%, or less than 1%.

3. The method of claim 1, wherein the interaction occurs at one or more residues of LRIG1 selected from region 245-260, wherein the residue positions correspond to positions 245-260 of SEQ ID NO: 2.

4. The method of claim 1, wherein the interaction occurs at one or more residues of VISTA selected from region 78-90 or 68-92, wherein the residue positions correspond to positions 78-90 or 68-92 of SEQ ID NO: 4.

5. The method of claim 1, wherein the antibody binds to at least one amino acid residue within Peptide 54 or Peptide 61.

6. The method of claim 1, wherein the antibody comprises a kD of less than 1 nM, 1.2 nM, 2 nM, 5 nM, 10 nM, 13.5 nM, 15 nM, 20 nM, 25 nM, or 30 nM.

7. The method of claim 1, wherein the antibody comprises a humanized antibody.

8. The method of any one of the claims 1-7, wherein the antibody comprises a full-length antibody or a binding fragment thereof.

9. The method of any one of the claims 1-8, wherein the antibody comprises a bispecific antibody or a binding fragment thereof.

10. The method of any one of the claims 1-9, wherein the antibody comprises a monovalent Fab′, a divalent Fab2, a single-chain variable fragment (scFv), a diabody, a minibody, a nanobody, a single-domain antibody (sdAb), or a camelid antibody or binding fragment thereof.

11. The method of claim 1, wherein the antibody is a humanized antibody comprising six complementarity-determining regions (CDRs) SEQ ID NOs: 81-86.

12. The method of claim 11, wherein the humanized antibody comprises a heavy chain variable region (VH) selected from SEQ ID NOs: 87 and 88.

13. The method of claim 11, wherein the humanized antibody comprises a light chain variable region (VL) selected from SEQ ID NOs: 89 and 90.

14. The method of claim 1, wherein the antibody is mab2, mab4, mab5, or mab6.

15. The method of any one of the claims 1-10, wherein the antibody comprises an IgG framework.

16. The method of any one of the claims 1-15, wherein the antibody comprises an IgG1, IgG2, or IgG4 framework.

17. A method of inducing immune activation, comprising:
contacting a plurality of cells comprising a LRIG1-expressing cell with an antibody under conditions to effect production of a cytokine, thereby inducing immune activation, wherein the antibody specifically binds to LRIG1.

18. The method of claim 17, wherein the plurality of cells further comprises a VISTA expressing cell.

19. The method of claim 18, wherein the anti-LRIG1 antibody further inhibits or disrupts an interaction of LRIG1 and VISTA.

20. The method of claim 19, wherein the LRIG1-VISTA interaction is reduced to less than 80%, less than 78%, less than 70%, less than 72%, less than 66%, less than 60%, less than 56%, less than 54%, less than 52%, less than 50%, less than 44%, less than 43%, less than 40%, less than 30%, less than 29%, less than 27%, less than 21%, less than 20%, less than 19%, less than 17%, less than 10%, less than 5%, or less than 1%.

21. The method of claim 19, wherein the interaction occurs at one or more residues of LRIG1 selected from region 245-260, wherein the residue positions correspond to positions 245-260 of SEQ ID NO: 2.

22. The method of claim 19, wherein the interaction occurs at one or more residues of VISTA selected from region 78-90 or 68-92, wherein the residue positions correspond to positions 78-90 or 68-92 of SEQ ID NO: 4.

23. The method of any one of the claims 17-22, wherein the antibody binds to at least one amino acid residue within Peptide 54 or Peptide 61.

24. The method of any one of the claims 17-23, wherein the antibody comprises a kD of less than 1 nM, 1.2 nM, 2 nM, 5 nM, 10 nM, 13.5 nM, 15 nM, 20 nM, 25 nM, or 30 nM.

25. The method of any one of the claims 17-24, wherein the antibody comprises a humanized antibody.

26. The method of any one of the claims 17-25, wherein the antibody comprises a full-length antibody or a binding fragment thereof.

27. The method of any one of the claims 17-26, wherein the antibody comprises a bispecific antibody or a binding fragment thereof.

28. The method of any one of the claims 17-27, wherein the antibody comprises a monovalent Fab′, a divalent Fab2, a single-chain variable fragment (scFv), a diabody, a minibody, a nanobody, a single-domain antibody (sdAb), or a camelid antibody or binding fragment thereof.

29. The method of any one of the claims 17-28, wherein the antibody is a humanized antibody comprising six complementarity-determining regions (CDRs) SEQ ID NOs: 81-86.

30. The method of any one of the claims 17-29, wherein the humanized antibody comprises a heavy chain variable region (VH) selected from SEQ ID NOs: 87 and 88.

31. The method of any one of the claims 17-30, wherein the humanized antibody comprises a light chain variable region (VL) selected from SEQ ID NOs: 89 and 90.

32. The method of any one of the claims 17-31, wherein the antibody is mab2, mab4, mab5, or mab6.

33. The method of any one of the claims 17-32, wherein the antibody comprises an IgG framework.

34. The method of any one of the claims 17-33, wherein the antibody comprises an IgG1, IgG2, or IgG4 framework.

35. The method of any one of claims 17-34, wherein the cytokine is an interferon.

36. The method of claim 35, wherein the interferon is IFNγ.

37. The method of claim 36, wherein the antibody results in IFNγ production higher than an isotype antibody.

38. The method of any one of the claims 17-37, wherein the immune activation comprises a proliferation of CD3+T lymphocytes, CD4+T helper cells, CD8+ cytotoxic T cells, B cells, Natural Killer cells, or a combination thereof.

39. The method of any one of the claims 17-38, wherein the immune activation comprises an increase in M1 macrophage population within the plurality of cells.

40. The method of any one of the claims 17-39, wherein the immune activation comprises a decrease in M2 macrophage population within the plurality of cells.

41. A method of reducing tumor cells within a tumor microenvironment (TME) in a subject, comprising
contacting a plurality of cells located within the TME with an antibody that specifically binds to LRIG1.

42. The method of claim 41, wherein the tumor cells are reduced by at least 5%, 10%, 15%, 20%, 25%, 30%, 35%, 40%, 45%, 50%, 60%, 70%, 80%, or 90%.

43. The method of claim 41, wherein the subject is diagnosed with a cancer.

44. The method of claim 43, wherein the cancer is a solid tumor.

45. The method of claim 44, wherein the cancer is breast cancer, colorectal cancer, kidney cancer, liver cancer, or lung cancer.

46. The method of claim 43, wherein the cancer is a hematologic malignancy.

47. The method of any one of the claims 43-46, wherein the cancer is a metastatic cancer.

48. The method of any one of the claims 43-46, wherein the cancer is a relapsed or refractory cancer.

49. The method of any one of the claims 41-48, wherein the antibody is formulated for systemic administration.

50. The method of any one of the claims 41-48, wherein the antibody is formulated for parenteral administration.

51. The method of any one of the claims 41-50, wherein the antibody is administered in combination with an additional therapeutic agent.

52. The method of claim 51, wherein the antibody and the additional therapeutic agent are administered simultaneously.

53. The method of claim 51, wherein the antibody and the additional therapeutic agent are administered sequentially.

54. The method of claim 53, wherein the antibody is administered prior to administering the additional therapeutic agent.

55. The method of claim 53, wherein the antibody is administered after administering the additional therapeutic agent.

56. The method of any one of the claims 51-55, wherein the additional therapeutic agent comprises an immune checkpoint modulator.

57. The method of any one of the claims 51-55, wherein the additional therapeutic agent comprises a chemotherapeutic agent, targeted therapeutic agent, hormonal therapeutic agent, or a stem cell-based therapeutic agent.

58. The method of claim 57, wherein the antibody is administered either prior to or after surgery.

59. The method of claim 57, wherein the antibody is administered in conjunction with, before, or after radiation therapy.

60. The method of any one of the claims 43-59, wherein the anti-LRIG1 antibody further inhibits or disrupts an interaction of LRIG1 and VISTA.

61. The method of claim 60, wherein the LRIG1-VISTA interaction is reduced to less than 80%, less than 78%, less than 70%, less than 72%, less than 66%, less than 60%, less than 56%, less than 54%, less than 52%, less than 50%, less than 44%, less than 43%, less than 40%, less than 30%, less than 29%, less than 27%, less than 21%, less than 20%, less than 19%, less than 17%, less than 10%, less than 5%, or less than 1%.

62. The method of claim 60, wherein the interaction occurs at one or more residues of LRIG1 selected from region 245-260, wherein the residue positions correspond to positions 245-260 of SEQ ID NO: 2.

63. The method of claim 60, wherein the interaction occurs at one or more residues of VISTA selected from region 78-90 or 68-92, wherein the residue positions correspond to positions 78-90 or 68-92 of SEQ ID NO: 4.

64. The method of any one of the claims 41-63, wherein the antibody binds to at least one amino acid residue within Peptide 54 or Peptide 61.

65. The method of any one of the claims 41-64, wherein the antibody comprises a kD of less than 1 nM, 1.2 nM, 2 nM, 5 nM, 10 nM, 13.5 nM, 15 nM, 20 nM, 25 nM, or 30 nM.

66. The method of any one of the claims 41-65, wherein the antibody comprises a humanized antibody.

67. The method of any one of the claims 41-66, wherein the antibody comprises a full-length antibody or a binding fragment thereof.

68. The method of any one of the claims 41-67, wherein the antibody comprises a bispecific antibody or a binding fragment thereof.

69. The method of any one of the claims 41-68, wherein the antibody comprises a monovalent Fab′, a divalent Fab2, a single-chain variable fragment (scFv), a diabody, a minibody, a nanobody, a single-domain antibody (sdAb), or a camelid antibody or binding fragment thereof.

70. The method of any one of the claims 41-69, wherein the antibody is a humanized antibody comprising six complementarity-determining regions (CDRs) SEQ ID NOs: 81-86.

71. The method of any one of the claims 41-70, wherein the humanized antibody comprises a heavy chain variable region (VH) selected from SEQ ID NOs: 87 and 88.

72. The method of any one of the claims 41-71, wherein the humanized antibody comprises a light chain variable region (VL) selected from SEQ ID NOs: 89 and 90.

73. The method of any one of the claims 41-72, wherein the antibody is mab2, mab4, mab5, or mab6.

74. The method of any one of the claims 41-73, wherein the antibody comprises an IgG framework.

75. The method of any one of the claims 41-74, wherein the antibody comprises an IgG1, IgG2, or IgG4 framework.

76. The method of any one of claims 41-75, further comprising inducing immune activation.

77. The method of claim 76, wherein the immune activation comprises production of a cytokine.

78. The method of claim 77, wherein the cytokine is an interferon, optionally IFNγ.

79. The method of any one of the claims 76-78, wherein the immune activation comprises a proliferation of CD3+T lymphocytes, CD4+T helper cells, CD8+ cytotoxic T cells, B cells, Natural Killer cells, or a combination thereof.

80. The method of any one of the claims 76-79, wherein the immune activation comprises an increase in M1 macrophage population within the plurality of cells.

81. The method of any one of the claims 76-80, wherein the immune activation comprises a decrease in M2 macrophage population within the plurality of cells.

82. The method of any one of the claims 41-81, wherein the subject is a human.


SEQUENCE LISTING (99 sequences)

SEQ ID NO: 1 - DNA, 4812 bp, Homo sapiens
gcgctccagacaagatggcgcggccggtccggggagggctcggggccccgcgccgctcgccttgccttctccttctctggctgcttttgcttcggctggagccggtgaccgccgcggccggcccgcgggcgccctgcgcggccgcctgcacttgcgctggggactcgctggactgcggtgggcgcgggctggctgcgttgcccggggacctgccctcctggacgcggagcctaaacctgagttacaacaaactctctgagattgaccctgctggttttgaggacttgccgaacctacaggaagtgtacctcaataataatgagttgacagcggtaccatccctgggcgctgcttcatcacatgtcgtctctctctttctgcagcacaacaagattcgcagcgtggaggggagccagctgaaggcctacctttccttagaagtgttagatctgagtttgaacaacatcacggaagtgcggaacacctgctttccacacggaccgcctataaaggagctcaacctggcaggcaatcggattggcaccctggagttgggagcatttgatggtctgtcacggtcgctgctaactcttcgcctgagcaaaaacaggatcacccagcttcctgtaagagcattcaagctacccaggctgacacaactggacctcaatcggaacaggattcggctgatagagggcctcaccttccaggggctcaacagcttggaggtgctgaagcttcagcgaaacaacatcagcaaactgacagatggggccttctggggactgtccaagatgcatgtgctgcacctggagtacaacagcctggtagaagtgaacagcggctcgctctacggcctcacggccctgcatcagctccacctcagcaacaattccatcgctcgcattcaccgcaagggctggagcttctgccagaagctgcatgagttggtcctgtccttcaacaacctgacacggctggacgaggagagcctggccgagctgagcagcctgagtgtcctgcgtctcagccacaattccatcagccacattgcggagggtgccttcaagggactcaggagcctgcgagtcttggatctggaccataacgagatttcgggcacaatagaggacacgagcggcgccttctcagggctcgacagcctcagcaagctgactctgtttggaaacaagatcaagtctgtggctaagagagcattctcggggctggaaggcctggagcacctgaaccttggagggaatgcgatcagatctgtccagtttgatgcctttgtgaagatgaagaatcttaaagagctccatatcagcagcgacagcttcctgtgtgactgccagctgaagtggctgcccccgtggctaattggcaggatgctgcaggcctttgtgacagccacctgtgcccacccagaatcactgaagggtcagagcattttctctgtgccaccagagagtttcgtgtgcgatgacttcctgaagccacagatcatcacccagccagaaaccaccatggctatggtgggcaaggacatccggtttacatgctcagcagccagcagcagcagctcccccatgacctttgcctggaagaaagacaatgaagtcctgaccaatgcagacatggagaactttgtccacgtccacgcgcaggacggggaagtgatggagtacaccaccatcctgcacctccgtcaggtcactttcgggcacgagggccgctaccaatgtgtcatcaccaaccactttggctccacctattcacataaggccaggctcaccgtgaatgtgttgccatcattcaccaaaacgccccacgacataaccatccggaccaccaccatggcccgcctcgaatgtgctgccacaggtcacccaaaccctcagattgcctggcagaaggatggaggcacggatttccccgctgcccgtgagcgacgcatgcatgtcatgccggatgacgacgtgtttttcatcactgatgtgaaaatagatgacgcaggggtttacagctgtactgctcagaactcagccggttctatttcagctaatgccaccctgactgtcctagagaccccatccttggtggtccccttggaagaccgtgtggtatctgtgggagaaacagtggccctccaatgcaaagccacggggaaccctccgccccgcatcacctggttcaagggggaccgcccgctgagcctcactgagcggcaccacttgacccctgacaaccagctcctggtggttcagaacgtggtggcagaggatgcgggccgatatacctgtgagatgtccaacaccctgggcacggagcgagctcacagccagctgagcgtcctgcccgcagcaggctgcaggaaggatgggaccacggtaggcatcttcaccattgctgtcgtgagcagcatcgtcctgacgtcactggtctgggtgtgcatcatctaccagaccaggaagaagagtgaagagtacagtgtcaccaacacagatgaaaccgtcgtgccaccagatgttccaagctacctctcttctcaggggaccctttctgaccgacaagaaaccgtggtcaggaccgagggtggccctcaggccaatgggcacattgagagcaatggtgtgtgtccaagagatgcaagccactttccagagcccgacactcacagcgttgcctgcaggcagccaaagctctgtgctgggtctgcgtatcacaaagagccgtggaaagcgatggagaaagctgaagggacacctgggccacataagatggaacacggtggccgggtcgtatgcagtgactgcaacaccgaagtggactgttactccaggggacaagccttccacccccagcctgtgtccagagacagcgcacagccaagtgcgccaaatggcccggagccgggtgggagtgaccaagagcattctccacatcaccagtgcagcaggactgccgctgggtcctgccccgagtgccaagggtcgctctaccccagtaaccacgatagaatgctgacggctgtgaagaaaaagccaatggcatctctagatgggaaaggggattcttcctggactttagcaaggttgtatcacccggactccacagagctacagcctgcatcttcattaacttcaggcagtccagagcgcgcggaagcccagtacttgcttgtttccaatggccacctccccaaagcatgtgacgccagtcccgagtccacgccactgacaggacagctccccgggaaacagagggtgccactgctgttggcaccaaaaagctaggttttgtctacctcagttcttgtcataccaatctctacgggaaagagaggtaggagaggctgcgaggaagcttgggttcaagcgtcactcatctgtacatagttgtaactcccatgtggagtatcagtcgctcacaggacttggatctgaagcacagtaaacgcaagaggggatttgtgtacaaaaggcaaaaaaagtatttgatatcattgtacataagagttttcagagatttcatatatatcttttacagaggctattttaatctttagtgcatggttaacagaaaaaaattatacaattttgacaatattatttttcgtatcaggttgctgtttaattttggagggggtggggaaatagttctggtgccttaacgcatggctggaatttatagaggctacaaccacatttgttcacaggagtttttggtgcggggtgggaaggatggaaggccttggatttatattgcacttcatagacccctaggctgctgtgcggtgggactccacatgcgccggaaggagcttcaggtgagcactgctcatgtgtggatgcccctgcaacaggcttccctgtctgtagagccaggggtgcaagtgccatccacacttgcagtgaatggcttttccttttaggtttaagtcctgtctgtctgtaaggcgtagaatctgtccgtctgtaaggcgtagaatgagggttgttaatccatcacaagcaaaaggtcagaacagttaaacactgcctttcctcctcctcttattttatgataaaagcaaatgtggccttctcagtatcattcgattgctatttgagacttttaaattaaggtaaaggctgctggtgttggtacctgtggatttttctatactgatgttttcgttttgccaatataatgagtattacattggccttgggggacagaaaggaggaagttctgacttttcagggctaccttatttctactaaggacccagagcaggcctgtccatgccattccttcgcacagatgaaactgagctgggactggaaaggacagcccttgacctgggttctgggtataatttgcacttttgagactggtagctaaccatcttatgagtgccaatgtgtcatttagtaaaacttaaatagaaacaaggtccttcaaatgttcctttggccaaaagctgaagggagttactgagaaaatagttaacaattactgtcaggtgtcatcactgttcaaaaggtaagcacatttagaattttgttcttgacagttaactgactaatcttacttccacaaaatatgtgaatttgctgcttctgagaggcaatgtgaaagagggagtattacttttatgtacaaagttatttatttatagaaattttggtacagtgtacattgaaaaccatgtaaaatattgaagtgtctaacaaatggcattgaagtgtctttaataaaggttcatttataaatgtcaaaaaaaaaaaaaaaaaaaaaaaaaaaaaaaaaaaaaaaaaaaaaaaaaaa

SEQ ID NO: 2 - protein, 1093 aa, Homo sapiens
MARPVRGGLGAPRRSPCLLLLWLLLLRLEPVTAAAGPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTMARLECAATGHPNPQIAWQKDGGTDFPAARERRMHVMPDDDVFFITDVKIDDAGVYSCTAQNSAGSISANATLTVLETPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVGIFTIAVVSSIVLTSLVWVCIIYQTRKKSEEYSVTNTDETVVPPDVPSYLSSQGTLSDRQETVVRTEGGPQANGHIESNGVCPRDASHFPEPDTHSVACRQPKLCAGSAYHKEPWKAMEKAEGTPGPHKMEHGGRVVCSDCNTEVDCYSRGQAFHPQPVSRDSAQPSAPNGPEPGGSDQEHSPHHQCSRTAAGSCPECQGSLYPSNHDRMLTAVKKKPMASLDGKGDSSWTLARLYHPDSTELQPASSLTSGSPERAEAQYLLVSNGHLPKACDASPESTPLTGQLPGKQRVPLLLAPKS

SEQ ID NO: 3 - DNA, 4774 bp, Homo sapiens
gggggcgggtgcctggagcacggcgctggggccgcccgcagcgctcactcgctcgcactcagtcgcgggaggcttccccgcgccggccgcgtcccgcccgctccccggcaccagaagttcctctgcgcgtccgacggcgacatgggcgtccccacggccctggaggccggcagctggcgctggggatccctgctcttcgctctcttcctggctgcgtccctaggtccggtggcagccttcaaggtcgccacgccgtattccctgtatgtctgtcccgaggggcagaacgtcaccctcacctgcaggctcttgggccctgtggacaaagggcacgatgtgaccttctacaagacgtggtaccgcagctcgaggggcgaggtgcagacctgctcagagcgccggcccatccgcaacctcacgttccaggaccttcacctgcaccatggaggccaccaggctgccaacaccagccacgacctggctcagcgccacgggctggagtcggcctccgaccaccatggcaacttctccatcaccatgcgcaacctgaccctgctggatagcggcctctactgctgcctggtggtggagatcaggcaccaccactcggagcacagggtccatggtgccatggagctgcaggtgcagacaggcaaagatgcaccatccaactgtgtggtgtacccatcctcctcccaggatagtgaaaacatcacggctgcagccctggctacgggtgcctgcatcgtaggaatcctctgcctccccctcatcctgctcctggtctacaagcaaaggcaggcagcctccaaccgccgtgcccaggagctggtgcggatggacagcaacattcaagggattgaaaaccccggctttgaagcctcaccacctgcccaggggatacccgaggccaaagtcaggcaccccctgtcctatgtggcccagcggcagccttctgagtctgggcggcatctgctttcggagcccagcacccccctgtctcctccaggccccggagacgtcttcttcccatccctggaccctgtccctgactctccaaactttgaggtcatctagcccagctgggggacagtgggctgttgtggctgggtctggggcaggtgcatttgagccagggctggctctgtgagtggcctccttggcctcggccctggttccctccctcctgctctgggctcagatactgtgacatcccagaagcccagcccctcaacccctctggatgctacatggggatgctggacggctcagcccctgttccaaggattttggggtgctgagattctcccctagagacctgaaattcaccagctacagatgccaaatgacttacatcttaagaagtctcagaacgtccagcccttcagcagctctcgttctgagacatgagccttgggatgtggcagcatcagtgggacaagatggacactgggccaccctcccaggcaccagacacagggcacggtggagagacttctcccccgtggccgccttggctcccccgttttgcccgaggctgctcttctgtcagacttcctctttgtaccacagtggctctggggccaggcctgcctgcccactggccatcgccaccttccccagctgcctcctaccagcagtttctctgaagatctgtcaacaggttaagtcaatctggggcttccactgcctgcattccagtccccagagcttggtggtcccgaaacgggaagtacatattggggcatggtggcctccgtgagcaaatggtgtcttgggcaatctgaggccaggacagatgttgccccacccactggagatggtgctgagggaggtgggtggggccttctgggaaggtgagtggagaggggcacctgccccccgccctccccatcccctactcccactgctcagcgcgggccattgcaagggtgccacacaatgtcttgtccaccctgggacacttctgagtatgaagcgggatgctattaaaaactacatggggaaacaggtgcaaaccctggagatggattgtaagagccagtttaaatctgcactctgctgctcctcccccacccccaccttccactccatacaatctgggcctggtggagtcttcgcttcagagccattcggccaggtgcgggtgatgttcccatctcctgcttgtgggcatgccctggctttgtttttatacacataggcaaggtgagtcctctgtggaattgtgattgaaggattttaaagcaggggaggagagtagggggcatctctgtacactctgggggtaaaacagggaaggcagtgcctgagcatggggacaggtgaggtggggctgggcagaccccctgtagcgtttagcaggatgggggccccaggtactgtggagagcatagtccagcctgggcatttgtctcctagcagcctacactggctctgctgagctgggcctgggtgctgaaagccaggatttggggctaggcgggaagatgttcgcccaattgcttggggggttggggggatggaaaaggggagcacctctaggctgcctggcagcagtgagccctgggcctgtggctacagccagggaaccccacctggacacatggccctgcttctaagccccccagttaggcccaaaggaatggtccactgagggcctcctgctctgcctgggctgggccaggggctttgaggagagggtaaacataggcccggagatggggctgacacctcgagtggccagaatatgcccaaaccccggcttctcccttgtccctaggcagaggggggtcccttcttttgttccctctggtcaccacaatgcttgatgccagctgccataggaagagggtgctggctggccatggtggcacacacctgtcctcccagcactttgcagggctgaggtggaaggaccgcttaagcccaggtgttcaaggctgctgtgagctgtgttcgagccactacactccagcctggggacggagcaaaactttgcctcaaaacaaattttaaaaagaaagaaagaaggaaagagggtatgtttttcacaattcatgggggcctgcatggcaggagtggggacaggacacctgctgttcctggagtcgaaggacaagcccacagcccagattccggttctcccaactcaggaagagcatgccctgccctctggggaggctggcctggccccagccctcagctgctgaccttgaggcagagacaacttctaagaatttggctgccagaccccaggcctggctgctgctgtgtggagagggaggcggcccgcagcagaacagccaccgcacttcctcctcagcttcctctggtgcggccctgccctctcttctctggacccttttacaactgaacgcatctgggcttcgtggtttcctgttttcagcgaaatttactctgagctcccagttccatcttcatccatggccacaggccctgcctacaacgcactagggacgtccctccctgctgctgctggggaggggcaggctgctggagccgccctctgagttgcccgggatggtagtgcctctgatgccagccctggtggctgtgggctggggtgcatgggagagctgggtgcgagaacatggcgcctccagggggcgggaggagcactaggggctggggcaggaggctcctggagcgctggattcgtggcacagtctgaggccctgagagggaaatccatgcttttaagaactaattcattgttaggagatcaatcaggaattaggggccatcttacctatctcctgacattcacagtttaatagagacttcctgcctttattccctcccagggagaggctgaaggaatggaattgaaagcaccatttggagggttttgctgacacagcggggactgctcagcactccctaaaaacacaccatggaggccactggtgactgctggtgggcaggctggccctgcctgggggagtccgtggcgatgggcgctggggtggaggtgcaggagccccaggacctgcttttcaaaagacttctgcctgaccagagctcccactacatgcagtggcccagggcagaggggctgatacatggcctttttcagggggtgctcctcgcggggtggacttgggagtgtgcagtgggacagggggctgcaggggtcctgccaccaccgagcaccaacttggcccctggggtcctgcctcatgaatgaggccttccccagggctggcctgactgtgctgggggctgggttaacgttttctcagggaaccacaatgcacgaaagaggaactggggttgctaaccaggatgctgggaacaaaggcctcttgaagcccagccacagcccagctgagcatgaggcccagcccatagacggcacaggccacctggcccattccctgggcattccctgctttgcattgctgcttctcttcaccccatggaggctatgtcaccctaactatcctggaatgtgttgagagggattctgaatgatcaatatagcttggtgagacagtgccgagatagatagccatgtctgccttgggcacgggagagggaagtggcagcatgcatgctgtttcttggccttttctgttagaatacttggtgctttccaacacactttcacatgtgttgtaacttgtttgatccacccccttccctgaaaatcctgggaggttttattgctgccatttaacacagagggcaatagaggttctgaaaggtctgtgtcttgtcaaaacaagtaaacggtggaactacgactaaa

SEQ ID NO: 4 - protein, 311 aa, Homo sapiens
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 5 - protein, 20 aa, Homo sapiens
AGPRAPCAAACTCAGDSLDC

SEQ ID NO: 6 - protein, 20 aa, Homo sapiens
CTCAGDSLDCGGRGLAALPG

SEQ ID NO: 7 - protein, 20 aa, Homo sapiens
GGRGLAALPGDLPSWTRSLN

SEQ ID NO: 8 - protein, 20 aa, Homo sapiens
DLPSWTRSLNLSYNKLSEID

SEQ ID NO: 9 - protein, 20 aa, Homo sapiens
LSYNKLSEIDPAGFEDLPNL

SEQ ID NO: 10 - protein, 20 aa, Homo sapiens
PAGFEDLPNLQEVYLNNNEL

SEQ ID NO: 11 - protein, 20 aa, Homo sapiens
QEVYLNNNELTAVPSLGAAS

SEQ ID NO: 12 - protein, 20 aa, Homo sapiens
TAVPSLGAASSHVVSLFLQH

SEQ ID NO: 13 - protein, 20 aa, Homo sapiens
SHVVSLFLQHNKIRSVEGSQ

SEQ ID NO: 14 - protein, 20 aa, Homo sapiens
NKIRSVEGSQLKAYLSLEVL

SEQ ID NO: 15 - protein, 20 aa, Homo sapiens
LKAYLSLEVLDLSLNNITEV

SEQ ID NO: 16 - protein, 20 aa, Homo sapiens
DLSLNNITEVRNTCFPHGPP

SEQ ID NO: 17 - protein, 20 aa, Homo sapiens
RNTCFPHGPPIKELNLAGNR

SEQ ID NO: 18 - protein, 20 aa, Homo sapiens
IKELNLAGNRIGTLELGAFD

SEQ ID NO: 19 - protein, 20 aa, Homo sapiens
IGTLELGAFDGLSRSLLTLR

SEQ ID NO: 20 - protein, 20 aa, Homo sapiens
GLSRSLLTLRLSKNRITQLP

SEQ ID NO: 21 - protein, 20 aa, Homo sapiens
LSKNRITQLPVRAFKLPRLT

SEQ ID NO: 22 - protein, 20 aa, Homo sapiens
VRAFKLPRLTQLDLNRNRIR

SEQ ID NO: 23 - protein, 20 aa, Homo sapiens
QLDLNRNRIRLIEGLTFQGL

SEQ ID NO: 24 - protein, 20 aa, Homo sapiens
LIEGLTFQGLNSLEVLKLQR

SEQ ID NO: 25 - protein, 20 aa, Homo sapiens
NSLEVLKLQRNNISKLTDGA

SEQ ID NO: 26 - protein, 20 aa, Homo sapiens
NNISKLTDGAFWGLSKMHVL

SEQ ID NO: 27 - protein, 20 aa, Homo sapiens
FWGLSKMHVLHLEYNSLVEV

SEQ ID NO: 28 - protein, 20 aa, Homo sapiens
HLEYNSLVEVNSGSLYGLTA

SEQ ID NO: 29 - protein, 20 aa, Homo sapiens
NSGSLYGLTALHQLHLSNNS

SEQ ID NO: 30 - protein, 20 aa, Homo sapiens
LHQLHLSNNSIARIHRKGWS

SEQ ID NO: 31 - protein, 20 aa, Homo sapiens
IARIHRKGWSFCQKLHELVL

SEQ ID NO: 32 - protein, 20 aa, Homo sapiens
FCQKLHELVLSFNNLTRLDE

SEQ ID NO: 33 - protein, 20 aa, Homo sapiens
SFNNLTRLDEESLAELSSLS

SEQ ID NO: 34 - protein, 20 aa, Homo sapiens
ESLAELSSLSVLRLSHNSIS

SEQ ID NO: 35 - protein, 20 aa, Homo sapiens
VLRLSHNSISHIAEGAFKGL

SEQ ID NO: 36 - protein, 20 aa, Homo sapiens
HIAEGAFKGLRSLRVLDLDH

SEQ ID NO: 37 - protein, 20 aa, Homo sapiens
RSLRVLDLDHNEISGTIEDT

SEQ ID NO: 38 - protein, 20 aa, Homo sapiens
NEISGTIEDTSGAFSGLDSL

SEQ ID NO: 39 - protein, 20 aa, Homo sapiens
SGAFSGLDSLSKLTLFGNKI

SEQ ID NO: 40 - protein, 20 aa, Homo sapiens
SKLTLFGNKIKSVAKRAFSG

SEQ ID NO: 41 - protein, 20 aa, Homo sapiens
KSVAKRAFSGLEGLEHLNLG

SEQ ID NO: 42 - protein, 20 aa, Homo sapiens
LEGLEHLNLGGNAIRSVQFD

SEQ ID NO: 43 - protein, 20 aa, Homo sapiens
GNAIRSVQFDAFVKMKNLKE

SEQ ID NO: 44 - protein, 20 aa, Homo sapiens
AFVKMKNLKELHISSDSFLC

SEQ ID NO: 45 - protein, 20 aa, Homo sapiens
LHISSDSFLCDCQLKWLPPW

SEQ ID NO: 46 - protein, 20 aa, Homo sapiens
DCQLKWLPPWLIGRMLQAFV

SEQ ID NO: 47 - protein, 20 aa, Homo sapiens
LIGRMLQAFVTATCAHPESL

SEQ ID NO: 48 - protein, 20 aa, Homo sapiens
TATCAHPESLKGQSIFSVPP

SEQ ID NO: 49 - protein, 20 aa, Homo sapiens
KGQSIFSVPPESFVCDDFLK

SEQ ID NO: 50 - protein, 20 aa, Homo sapiens
ESFVCDDFLKPQIITQPETT

SEQ ID NO: 51 - protein, 20 aa, Homo sapiens
PQIITQPETTMAMVGKDIRF

SEQ ID NO: 52 - protein, 20 aa, Homo sapiens
MAMVGKDIRFTCSAASSSSS

SEQ ID NO: 53 - protein, 20 aa, Homo sapiens
TCSAASSSSSPMTFAWKKDN

SEQ ID NO: 54 - protein, 20 aa, Homo sapiens
PMTFAWKKDNEVLTNADMEN

SEQ ID NO: 55 - protein, 20 aa, Homo sapiens
EVLTNADMENFVHVHAQDGE

SEQ ID NO: 56 - protein, 20 aa, Homo sapiens
FVHVHAQDGEVMEYTTILHL

SEQ ID NO: 57 - protein, 20 aa, Homo sapiens
VMEYTTILHLRQVTFGHEGR

SEQ ID NO: 58 - protein, 20 aa, Homo sapiens
RQVTFGHEGRYQCVITNHFG

SEQ ID NO: 59 - protein, 20 aa, Homo sapiens
YQCVITNHFGSTYSHKARLT

SEQ ID NO: 60 - protein, 20 aa, Homo sapiens
STYSHKARLTVNVLPSFTKT

SEQ ID NO: 61 - protein, 20 aa, Homo sapiens
VNVLPSFTKTPHDITIRTTT

SEQ ID NO: 62 - protein, 20 aa, Homo sapiens
PHDITIRTTTMARLECAATG

SEQ ID NO: 63 - protein, 20 aa, Homo sapiens
MARLECAATGHPNPQIAWQK

SEQ ID NO: 64 - protein, 20 aa, Homo sapiens
HPNPQIAWQKDGGTDFPAAR

SEQ ID NO: 65 - protein, 20 aa, Homo sapiens
DGGTDFPAARERRMHVMPDD

SEQ ID NO: 66 - protein, 20 aa, Homo sapiens
ERRMHVMPDDDVFFITDVKI

SEQ ID NO: 67 - protein, 20 aa, Homo sapiens
DVFFITDVKIDDAGVYSCTA

SEQ ID NO: 68 - protein, 20 aa, Homo sapiens
DDAGVYSCTAQNSAGSISAN

SEQ ID NO: 69 - protein, 20 aa, Homo sapiens
QNSAGSISANATLTVLETPS

SEQ ID NO: 70 - protein, 20 aa, Homo sapiens
ATLTVLETPSLVVPLEDRVV

SEQ ID NO: 71 - protein, 20 aa, Homo sapiens
LVVPLEDRVVSVGETVALQC

SEQ ID NO: 72 - protein, 20 aa, Homo sapiens
SVGETVALQCKATGNPPPRI

SEQ ID NO: 73 - protein, 20 aa, Homo sapiens
KATGNPPPRITWFKGDRPLS

SEQ ID NO: 74 - protein, 20 aa, Homo sapiens
TWFKGDRPLSLTERHHLTPD

SEQ ID NO: 75 - protein, 20 aa, Homo sapiens
LTERHHLTPDNQLLVVQNVV

SEQ ID NO: 76 - protein, 20 aa, Homo sapiens
NQLLVVQNVVAEDAGRYTCE

SEQ ID NO: 77 - protein, 20 aa, Homo sapiens
AEDAGRYTCEMSNTLGTERA

SEQ ID NO: 78 - protein, 20 aa, Homo sapiens
MSNTLGTERAHSQLSVLPAA

SEQ ID NO: 79 - protein, 20 aa, Homo sapiens
HSQLSVLPAAGCRKDGTTVG

SEQ ID NO: 80 - protein, 10 aa, Homo sapiens
GCRKDGTTVG

SEQ ID NO: 81 - protein, 7 aa, Artificial Sequence
  Description of Artificial Sequence Synthetic peptide
GYTFTSY

SEQ ID NO: 82 - protein, 12 aa, Artificial Sequence
  Description of Artificial Sequence Synthetic peptide
WIYPGNVNTKYN

SEQ ID NO: 83 - protein, 8 aa, Artificial Sequence
  Description of Artificial Sequence Synthetic peptide
EELGGFAY

SEQ ID NO: 84 - protein, 11 aa, Artificial Sequence
  Description of Artificial Sequence Synthetic peptide
RASQDISNYLS

SEQ ID NO: 85 - protein, 7 aa, Artificial Sequence
  Description of Artificial Sequence Synthetic peptide
YTSILHS

SEQ ID NO: 86 - protein, 9 aa, Artificial Sequence
  Description of Artificial Sequence Synthetic peptide
QQGNTLPRT

SEQ ID NO: 87 - protein, 117 aa, Artificial Sequence
  Description of Artificial Sequence Synthetic polypeptide
QVQLVQSGAEVKKPGASVKVSCKASGYTFTSYYLHWVRQAPGQRLEWMGWIYPGNVNTKYNQKFQGRVTITADKSASTAYMELSSLRSEDTAVYFCAREELGGFAYWGQGTLVTVSS

SEQ ID NO: 88 - protein, 117 aa, Artificial Sequence
  Description of Artificial Sequence Synthetic polypeptide
QVQLVQSGAEVKKPGASVKVSCKASGYTFTSYYLHWVRQAPGQRLEWIGWIYPGNVNTKYNEKFQGRVTLTADKSASTAYMELSSLRSEDTAVYFCAREELGGFAYWGQGTLVTVSS

SEQ ID NO: 89 - protein, 107 aa, Artificial Sequence
  Description of Artificial Sequence Synthetic polypeptide
DIQMTQSPSSVSASIGDRVTITCRASQDISNYLSWYQQKPGKAPKLLIYYTSILHSGVPSRFSGSGSGTDYTLTISSLQPEDFATYFCQQGNTLPRTFGGGTKVEIK

SEQ ID NO: 90 - protein, 106 aa, Artificial Sequence
  Description of Artificial Sequence Synthetic polypeptide
DIQMTQSPSSLSASVGNRVTITCRASQDISNYLSWYQQKPGKVPKLLIYYTSIHSGVPSRFSGSGSGTDYSLTISSLQPEDVATYFCQQGNTLPRTFGQGTKVEIK

SEQ ID NO: 91 - protein, 4 aa, Artificial Sequence
  Description of Artificial Sequence Synthetic peptide
GGFG

SEQ ID NO: 92 - protein, 4 aa, Artificial Sequence
  Description of Artificial Sequence Synthetic peptide
ALAL

SEQ ID NO: 93 - protein, 4 aa, Artificial Sequence
  Description of Artificial Sequence Synthetic peptide
GFLG

SEQ ID NO: 94 - protein, 21 aa, Homo sapiens
RNNISKLTDGAFWGLSKMHVL

SEQ ID NO: 95 - protein, 21 aa, Homo sapiens
WYRSSRGEVQTCSERRPIRNL

SEQ ID NO: 96 - protein, 13 aa, Homo sapiens
GEVQTCSERRPIR

SEQ ID NO: 97 - protein, 16 aa, Homo sapiens
NNISKLTDGAFWGLSK

SEQ ID NO: 98 - protein, 24 aa, Homo sapiens
FYKTWYRSSRGEVQTCSERRPIRN

SEQ ID NO: 99 - protein, 6 aa, Homo sapiens
FWGLSK